(19) United States
(12) Patent Application Publication
(10) Pub. No.: US 2011/0144107 A1
(43) Pub. Date: Jun. 16, 2011

(54) COMPOUNDS AND COMPOSITIONS USEFUL FOR THE TREATMENT OF MALARIA

(75) Inventors: Arnab K. Chatterjee, San Diego, CA (US); Advait Nagle, San Diego, CA (US); Tao Wu, San Diego, CA (US); Nathanael S. Gray, Boston, MA (US)

(73) Assignees: IRM LLC, Hamilton (BM); DANA-FARBER CANCER INSTITUTE INC., Boston, MA (US)

(21) Appl. No.: 12/997,242

(22) PCT Filed: Jun. 11, 2009
(86) PCT No.: PCT/US09/47074

§ 371 Date: Feb. 23, 2011

Related U.S. Application Data
(60) Provisional application No. 61/060,779, filed on Jun. 11, 2008.

(51) Int. Cl. A61K 31/5377; C07D 401/14; A61K 31/506; C07D 233/56; A61K 31/415; C07D 401/04; A61K 31/454; C07D 211/32; A61K 31/445; A61K 31/4965; C07D 241/04; C07D 417/14; C07D 413/04; C12N 5/02

(52) U.S. Cl. 514/235.5; 544/297; 514/275; 548/343.5; 514/396; 546/208; 514/326; 546/225; 514/331; 514/255.04; 544/403; 544/130; 435/375

(57) ABSTRACT

The invention provides a class of compounds of formula I, pharmaceutical compositions comprising such compounds and methods of using such compounds to treat or prevent malaria.

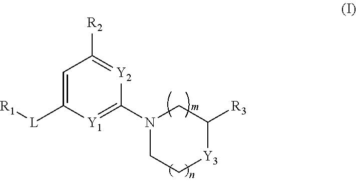

### CROSS-REFERENCE TO RELATED APPLICATIONS

This application claims the benefit of priority to U.S. Provisional Patent Application No. 61/060,779, filed 11 Jun. 2008. The full disclosure of this application is incorporated herein by reference in its entirety and for all purposes.

### BACKGROUND OF THE INVENTION

1. Field of the Invention

The invention provides a class of compounds, pharmaceutical compositions comprising such compounds and methods of using such compounds to treat or prevent malaria.

2. Background

Malaria is an infectious disease caused by four protozoan parasites: Plasmodium falciparum; Plasmodium vivax; Plasmodium ovale; and Plasmodium malaria. These four parasites are typically transmitted by the bite of an infected female Anopheles mosquito. Malaria is a problem in many parts of the world and over the last few decades the malaria burden has steadily increased. An estimated 1-3 million people die every year from malaria—mostly children under the age of 5. This increase in malaria mortality is due in part to the fact that Plasmodium falciparum, the deadliest malaria parasite, has acquired resistance against nearly all available antimalarial drugs, with the exception of the artemisinin derivatives.

Leishmaniasis is caused by one or more than 20 varieties of parasitic protozoa that belong to the genus Leishmania, and is transmitted by the bite of female sand flies. Leishmaniasis is endemic in about 88 countries, including many tropical and sub-tropical areas.

There are four main forms of Leishmaniasis. Visceral leishmaniasis, also called kala-azar, is the most serious form and is caused by the parasite Leishmania donovani. Patients who develop visceral leishmaniasis can die within months unless they receive treatment. The two main therapies for visceral leishmaniasis are the antimony derivatives sodium stibogluconate (Pentostam®) and meglumine antimoniate (Glucantim®). Sodium stibogluconate has been used for about 70 years and resistance to this drug is a growing problem. In addition, the treatment is relatively long and painful, and can cause undesirable side effects.

Human African Trypanosomiasis, also known as sleeping sickness, is a vector-borne parasitic disease. The parasites concerned are protozoa belonging to the Trypanosoma Genus. They are transmitted to humans by tsetse fly (Glossina Genus) bites which have acquired their infection from human beings or from animals harboring the human pathogenic parasites.

Chagas disease (also called American Trypanosomiasis) is another human parsitic disease that is endemic amongst poor populations on the American continent. The disease is caused by the protozoan parasite Trypanosoma cruzi, which is transmitted to humans by blood-sucking insects. The human disease occurs in two stages: the acute stage, which occurs shortly after infection and the chronic stage, which can develop over many years. Chronic infections result in various neurological disorders, including dementia, damage to the heart muscle and sometimes dilation of the digestive tract, as well as weight loss. Untreated, the chronic disease is often fatal.

The drugs currently available for treating Chagas disease are Nifurtimox and benznidazole. However, problems with these current therapies include their diverse side effects, the length of treatment, and the requirement for medical supervision during treatment. Furthermore, treatment is really only effective when given during the acute stage of the disease. Resistance to the two frontline drugs has already occurred. The antifungal agent Amphotericin b has been proposed as a second-line drug, but this drug is costly and relatively toxic.

In view of the foregoing, it is desirable to develop novel compounds as antiparasitic agents.

### SUMMARY OF THE INVENTION

In one aspect, the present invention provides a compound of Formula I:

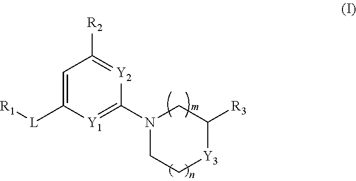

in which:

L is selected from —NR₄—, —NR₄S(O)₂—, —S(O)₂NR₄—, —C(O)O—, —OC(O)—, —C(O)—, —NR₄C(O)O—, —OC(O)NR₄—, —NR₄C(O)—, —C(O)NR₄—, —NR₄C(O)NR₄—, —NR₄NR₄C(O)— and —C(O)NR₄NR₄—; wherein R₄ is selected from hydrogen and —SO₂R₅; wherein R₅ is selected from hydrogen and C₁₋₆alkyl;

n and m are independently selected from 0 and 1;

R₁ is selected from C₁₋₆alkyl, C₆₋₁₀aryl-C₀₋₄alkyl, C₃₋₁₂cycloalkyl, 5-10 member heteroaryl and 3-8 member heterocycloalkyl; wherein said heteroaryl and heterocycloalkyl have up to 4 members selected from N, O and S(O)₀₋₂; wherein said aryl, heteroaryl, cycloalkyl or heterocycloalkyl of R₁ is optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy, —NR₆C(O)R₇, —C(O)NR₆R₇, —C(O)OR₇, —S(O)₂NR₆R₇, —S(O)₂R₇, C₆₋₁₀aryl, 3-8 member heterocycloalkyl-C₀₋₄alkyl and 5-10 member heteroaryl; wherein said heteroaryl and heterocycloalkyl have up to 4 members selected from N, O and S(O)₀₋₂; wherein R₆ is selected from hydrogen and C₁₋₆alkyl; and R₇ is selected from hydrogen, C₁₋₆alkyl and 5-10 member heteroaryl; wherein said heteroaryl has up to 4 members selected from N, O and S(O)₀₋₂; wherein said aryl, heterocycloalkyl or heteroaryl substituents of R₁ are optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy and 3-8 member heterocycloalkyl; wherein said heterocycloalkyl has up to 4 members selected from N, O and S(O)₀₋₂; wherein said alkyl substituents of R₁ are optionally substituted with —COOH;

R₂ is selected from hydrogen, halo, C₁₋₆alkyl, halo-substituted-C₁₋₆alkyl, C₁₋₆alkoxy and halo-substituted-C₁₋₆alkoxy;

R₃ is selected from hydrogen, C₁₋₆alkyl, C(O)NR₈R₉ and C(O)OR₉; wherein R₈ and R₉ are independently selected from hydrogen and C₁₋₆alkyl;

Y₁ and Y₂ are independently selected from CH and N;

Y₃ is selected from O, NR₁₀ and CR₁₀R₁₁; wherein R₁₀ and R₁₁ are independently selected from hydrogen, C₁₋₆alkyl, 3-8 member heterocycloalkyl, —NR₁₂R₁₃ and —NR₁₂C(O)OR₁₃; wherein said heterocycloalkyl has up to 4 members selected from N, O and S(O)₀₋₂; wherein said heterocycloalkyl of R₁₀ or R₁₁ is optionally substituted with 1 to 3 radicals independently selected from halo, C₁₋₆alkyl and halo-substituted-C₁₋₆alkyl; wherein R₁₂ and R₁₃ are independently selected from hydrogen and C₁₋₆alkyl; or R₃ and R₁₀ together with the carbon atoms to which R₃ and R₁₀ are attached from a phenyl ring (fused to the piperidinyl, for example compound 81 of table 1); and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which a compound of the invention can prevent, inhibit or ameliorate the pathology and/or symptomology of disease caused by a parasite (such as, for example, Plasmodium falciparum, Plasmodium vivax, Plasmodium ovale, Plasmodium malaria, Trypanosoma cruzi or a parasite of the Leishmania genus such as, for example, Leishmania donovani) which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease caused by a parasite in an animal. The disease may be malaria, leishmaniasis and/or Chagas disease.

In a fifth aspect, the present invention provides a process for preparing compounds of Formula I and the N-oxide derivatives, prodrug derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is as defined for aryl where one or more of the ring members are a heteroatom selected from N, O, C(O) and S(O)₀₋₂. For example 5-10 member heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, 3-8 member heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but may also be bromo or iodo.

“Treat”, “treating” and “treatment” refer to a method of alleviating or abating a disease and/or its attendant symptoms. In the present description, the term “treatment” includes both prophylactic or preventative treatment as well as curative or disease suppressive treatment, including treatment of patients at risk of contracting the disease or suspected to have contracted the disease as well as ill patients. This term further includes the treatment for the delay of progression of the disease.

### DESCRIPTION OF THE PREFERRED EMBODIMENTS

The invention provides a novel class of compounds, pharmaceutical compositions comprising such compounds and methods of using such compounds to treat or prevent diseases or disorders associated with a parasite. In particular, the compounds can be used to treat malaria, leishmaniasis and/or Chagas disease.

In one embodiment, with reference to compounds of Formula I:

L is selected from —NR₄—, —S(O)₂NR₄—, —OC(O)—, —OC(O)NR₄—, —NR₄C(O)—, —C(O)NR₄—, —C(O)—, —NR₄C(O)NR₄— and —NR₄NR₄C(O)—; wherein R₄ is selected from hydrogen and —SO₂R₅; wherein R₅ is selected from hydrogen and C₁₋₆alkyl;

n and m are independently selected from 0 and 1;

R₁ is selected from C₁₋₆alkyl, C₆₋₁₀aryl-C₀₋₄alkyl, C₃₋₁₂cycloalkyl, 5-10 member heteroaryl and 3-8 member heterocycloalkyl; wherein said heteroaryl and heterocycloalkyl have up to 4 members selected from N, O and S(O)₀₋₂; wherein said aryl, heteroaryl, cycloalkyl or heterocycloalkyl of R₁ is optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy, —NR₆C(O)R₇, —C(O)NR₆R₇, —C(O)OR₇, —S(O)₂NR₆R₇, —S(O)₂R₇, C₆₋₁₀aryl, 3-8 member heterocycloalkyl-C₀₋₄alkyl and 5-10 member heteroaryl; wherein said heteroaryl and heterocycloalkyl have up to 4 members selected from N, O and S(O)₀₋₂; wherein R₆ is selected from hydrogen and C₁₋₆alkyl; and R₇ is selected from hydrogen, C₁₋₆alkyl and 5-10 member heteroaryl; wherein said heteroaryl has up to 4 members selected from N, O and S(O)₀₋₂; wherein said aryl, heterocycloalkyl or heteroaryl substituents of R₁ are optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy and 3-8 member heterocycloalkyl; wherein said heterocycloalkyl has up to 4 members selected from N, O and S(O)₀₋₂; wherein said alkyl substituents of R₁ are optionally substituted with —COOH;

R₂ is selected from hydrogen, halo, C₁₋₆alkyl and halo-substituted-C₁₋₆alkyl;

R₃ is selected from hydrogen, C(O)NR₈R₉ and C(O)OR₉; wherein R₈ and R₉ are independently selected from hydrogen and C₁₋₆alkyl;

Y₁ and Y₂ are independently selected from CH and N;

Y₃ is selected from O, NR₁₀ and CR₁₀R₁₁; wherein R₁₀ and R₁₁ are independently selected from hydrogen, C₁₋₆alkyl, 3-8 member heterocycloalkyl, —NR₁₂R₁₃ and —NR₁₂C(O)OR₁₃; wherein said heterocycloalkyl has up to 4 members selected from N, O and S(O)₀₋₂; wherein said heterocycloalkyl of R₁₀ or R₁₁ is optionally substituted with 1 to 3 radicals independently selected from halo, C₁₋₆alkyl and halo-substituted-C₁₋₆alkyl; wherein R₁₂ and R₁₃ are independently selected from hydrogen and C₁₋₆alkyl; or R₃ and R₁₀ together with the carbon atoms to which R₃ and R₁₀ are attached from a phenyl ring.

In a further embodiment, R₁ is selected from methyl, propyl, phenyl, cyclopropyl, pyridinyl, thiazolyl, pyrimidinyl, indolin-1-yl, piperazinyl, benzyl, 1H-indazol-5-yl, 1H-benzo[d]imidazol-2-yl, imidazolyl, 1H-indol-5-yl, benzo[d]thiazol-2-yl and 4-methyl-2-oxo-1,2-dihydroquinolin-6-yl; wherein said phenyl, benzyl, cyclopropyl, pyridinyl, thiazolyl, N-thiazol-2-ylsulfamoyl, indolin-1-yl, piperazinyl, 1H-indol-5-yl, 1H-indazol-5-yl, 1H-benzo[d]imidazol-2-yl, imidazolyl, benzo[d]thiazol-2-yl or 4-methyl-2-oxo-1,2-dihydroquinolin-6-yl is optionally substituted with 1 to 3 radicals independently selected from halo, cyano, trifluoromethyl, trifluoromethoxy, methyl-carbonyl-amino, amino-carbonyl, methyl, t-butyl, methoxy, propyl-sulfonyl, piperazinyl-methyl, piperidinyl, pyrazolyl, morpholino, imidazolyl, 2-carboxypropan-2-yl, phenyl and ethoxy-carbonyl; wherein said phenyl, piperidinyl, pyrazolyl, morpholino, piperazinyl-methyl or imidazolyl substituents of R₁ are optionally substituted with a radical selected from methyl, trifluoromethyl and pyrrolidinyl.

In a further embodiment, R₂ is selected from hydrogen, chloro, fluoro, trifluoromethyl, methyl and t-butyl; and R₃ is selected from amino-carbonyl and ethoxy-carbonyl.

In a further embodiment, Y₃ is selected from O, NR₁₀ and CR₁₀R₁₁; wherein R₁₀ is selected from hydrogen and methyl; and R₁₁ is selected from dimethyl-amino, t-butoxy-carbonyl-amino, morpholino, pyrrolidinyl, piperidinyl, piperazinyl, 2-oxopyrrolidin-1-yl and 2-oxopiperidin-1-yl; wherein said morpholino, piperazinyl, pyrrolidinyl, piperidinyl, 2-oxopyrrolidin-1-yl or 2-oxopiperidin-1-yl is optionally substituted with a radical selected from halo and methyl.

In a further embodiment are compounds selected from: N-(methylsulfonyl)-N-(3-(4-(pyrrolidin-1-yl)piperidin-1-yl)-5-(trifluoromethyl)phenyl)methanesulfonamide; N-(3-(4-(pyrrolidin-1-yl)piperidin-1-yl)-5-(trifluoromethyl)phenyl)-3-(trifluoromethyl)benzenesulfonamide; 4-methyl-N-(3-(4-(pyrrolidin-1-yl)piperidin-1-yl)-5-(trifluoromethyl)phenyl)benzenesulfonamide; N-(3-(N-(3-(4-(pyrrolidin-1-yl)piperidin-1-yl)-5-(trifluoromethyl)phenyl)sulfamoyl)phenyl)acetamide; 4-tert-butyl-N-(3-(4-(pyrrolidin-1-yl)piperidin-1-yl)-5-(trifluoromethyl)phenyl)benzenesulfonamide; 4-methoxy-N-(3-(4-(pyrrolidin-1-yl)piperidin-1-yl)-5-(trifluoromethyl)phenyl)benzenesulfonamide; 3-chloro-N-(3-fluoro-5-(4-(pyrrolidin-1-yl)piperidin-1-yl)phenyl)benzamide; tert-butyl 3-(4-(pyrrolidin-1-yl)piperidin-1-yl)-5-(trifluoromethyl)phenylcarbamate; N-(3-(4-(pyrrolidin-1-yl)piperidin-1-yl)-5-(trifluoromethyl)phenyl)cyclopropanecarboxamide; 2-chloro-N-(3-(4-(pyrrolidin-1-yl)piperidin-1-yl)-5-(trifluoromethyl)phenyl)nicotinamide; 2-fluoro-N-(3-(4-(pyrrolidin-1-yl)piperidin-1-yl)-5-(trifluoromethyl)phenyl)benzamide; N-(3-fluoro-5-(4-(pyrrolidin-1-yl)piperidin-1-yl)phenyl)-3-(trifluoromethyl)benzamide; 2,4-dichloro-N-(3-(4-(pyrrolidin-1-yl)piperidin-1-yl)-5-(trifluoromethyl)phenyl)benzamide; 3-cyano-N-(3-(4-(pyrrolidin-1-yl)piperidin-1-yl)-5-(trifluoromethyl)phenyl)benzamide; 4-methoxy-N-(3-(4-(pyrrolidin-1-yl)piperidin-1-yl)-5-(trifluoromethyl)phenyl)benzamide; 3-methyl-N-(3-(4-(pyrrolidin-1-yl)piperidin-1-yl)-5-(trifluoromethyl)phenyl)benzamide; N-(3-(4-(pyrrolidin-1-yl)piperidin-1-yl)-5-(trifluoromethyl)phenyl)-3-(trifluoromethyl)benzamide; 3-fluoro-N-(3-(4-(pyrrolidin-1-yl)piperidin-1-yl)-5-(trifluoromethyl)phenyl)benzamide; 3-(4-(pyrrolidin-1-yl)piperidin-1-yl)-N-(3-(4-(pyrrolidin-1-yl)piperidin-1-yl)-5-(trifluoromethyl)phenyl)-5-(trifluoromethyl)benzamide; 1-(4-chloro-2-methylphenyl)-3-(3-(4-(pyrrolidin-1-yl)piperidin-1-yl)-5-(trifluoromethyl)phenyl)urea; 1-(3-(4-(pyrrolidin-1-yl)piperidin-1-yl)-5-(trifluoromethyl)phenyl)-3-(3-(trifluoromethyl)phenyl)urea; 1-(3-chlorophenyl)-3-(3-(4-(pyrrolidin-1-yl)piperidin-1-yl)-5-(trifluoromethyl)phenyl)urea; 1-(3-(4-methyl-1H-imidazol-1-yl)-5-(trifluoromethyl)phenyl)-3-(3-(4-(pyrrolidin-1-yl)piperidin-1-yl)-5-(trifluoromethyl)phenyl)urea; 1-(3,5-dichlorophenyl)-3-(3-(4-(pyrrolidin-1-yl)piperidin-1-yl)-5-(trifluoromethyl)phenyl)urea; 1,3-bis(3-(4-(pyrrolidin-1-yl)piperidin-1-yl)-5-(trifluoromethyl)phenyl)urea; 2-methyl-2-(4-(3-(4-methyl-1,4′-bipiperidin-1′-yl)-5-(trifluoromethyl)benzamido)phenyl)propanoic acid; 3-(4-(pyrrolidin-1-yl)piperidin-1-yl)-5-(trifluoromethyl)-N-(4-(trifluoromethyl)thiazol-2-yl)benzamide; 3-chloro-N-(3-morpholino-5-(trifluoromethyl)phenyl)benzamide; 3-(4-(pyrrolidin-1-yl)piperidin-1-yl)-N-(4-(N-thiazol-2-ylsulfamoyl)phenyl)-5-(trifluoromethyl)benzamide; 1-(3-(trifluoromethyl)-5-(3-(trifluoromethyl)phenylcarbamoyl)phenyl)piperidine-3-carboxamide; N-propyl-3-(4-(pyrrolidin-1-yl)piperidin-1-yl)-5-(trifluoromethyl)benzamide; N-(3-chlorophenyl)-N-methyl-3-(4-(pyrrolidin-1-yl)piperidin-1-yl)-5-(trifluoromethyl)benzamide; 3-(4-(pyrrolidin-1-yl)piperidin-1-yl)-5-(trifluoromethyl)-N-(6-(trifluoromethyl)pyrimidin-4-yl)benzamide; N-(2-chloropyridin-4-yl)-3-(4-(pyrrolidin-1-yl)piperidin-1-yl)-5-(trifluoromethyl)benzamide; 3-morpholino-5-(trifluoromethyl)-N-(3-(trifluoromethyl)phenyl)benzamide; N-(3-carbamoylphenyl)-3-(4-(pyrrolidin-1-yl)piperidin-1-yl)-5-(trifluoromethyl)benzamide; N-(4-(2-chlorophenyl)thiazol-2-yl)-3-(4-(pyrrolidin-1-yl)piperidin-1-yl)-5-(trifluoromethyl)benzamide; N′-(3-chlorophenyl)-3-(4-(pyrrolidin-1-yl)piperidin-1-yl)-5-(trifluoromethyl)benzohydrazide; 3-(4-(pyrrolidin-1-yl)piperidin-1-yl)-5-(trifluoromethyl)-N′-(3-(trifluoromethyl)phenyl)benzohydrazide; N-(3-chlorophenyl)-3-(piperidin-1-yl)-5-(trifluoromethyl)benzamide; N-(pyrimidin-4-yl)-3-(4-(pyrrolidin-1-yl)piperidin-1-yl)-5-(trifluoromethyl)benzamide; (3-(4-(pyrrolidin-1-yl)piperidin-1-yl)-5-(trifluoromethyl)phenyl)(6-(trifluoromethyl)indolin-1-yl)methanone; N-(2-chlorophenyl)-3-(4-(pyrrolidin-1-yl)piperidin-1-yl)-5-(trifluoromethyl)benzamide; N-(3-chlorophenyl)-3-(4-(pyrrolidin-1-yl)piperidin-1-yl)benzamide; 3-(trifluoromethyl)-N-(3-(trifluoromethyl)phenyl)-5-(443-(trifluoromethyl)phenyl)piperazin-1-yl)benzamide; 3-(piperidin-1-yl)-5-(trifluoromethyl)-N-(3-(trifluoromethyl)phenyl)benzamide; 3-(2-oxo-1,4′-bipiperidin-1′-yl)-5-(trifluoromethyl)-N-(3-(trifluoromethyl)phenyl)benzamide; N-(4-tert-butylthiazol-2-yl)-3-(4-(pyrrolidin-1-yl)piperidin-1-yl)-5-(trifluoromethyl)benzamide; (3-(4-(pyrrolidin-1-yl)piperidin-1-yl)-5-(trifluoromethyl)phenyl)(4-(3-(trifluoromethyl)phenyl)piperazin-1-yl)methanone; tert-butyl 1-(3-(trifluoromethyl)-5-(3-(trifluoromethyl)phenylcarbamoyl)phenyl)piperidin-4-ylcarbamate; N-(4,5-dimethylthiazol-2-yl)-3-(4-(pyrrolidin-1-yl)piperidin-1-yl)-5-(trifluoromethyl)benzamide; N-(3-chloro-4-methoxyphenyl)-3-(4-(pyrrolidin-1-yl)piperidin-1-yl)-5-(trifluoromethyl)benzamide; N-(4-morpholinophenyl)-3-(4-(pyrrolidin-1-yl)piperidin-1-yl)-5-(trifluoromethyl)benzamide; 3-(4-(2-oxopyrrolidin-1-yl)piperidin-1-yl)-5-(trifluoromethyl)-N-(3-(trifluoromethyl)phenyl)benzamide; N-(4-morpholinophenyl)-3-(4-(pyrrolidin-1-yl)piperidin-1-yl)-5-(trifluoromethyl)benzamide; 3-(4-(pyrrolidin-1-yl)piperidin-1-yl)-5-(trifluoromethyl)-N-(3-(trifluoromethyl)benzyl)benzamide; N-(3-(1H-pyrazol-4-yl)phenyl)-3-(4-(pyrrolidin-1-yl)piperidin-1-yl)-5-(trifluoromethyl)benzamide; N-(1H-indazol-5-yl)-3-(4-(pyrrolidin-1-yl)piperidin-1-yl)-5-(trifluoromethyl)benzamide; ethyl 1-(3-(trifluoromethyl)-5-(3-(trifluoromethyl)phenylcarbamoyl)phenyl)piperidine-3-carboxylate; N-phenyl-3-(4-(pyrrolidin-1-yl)piperidin-1-yl)-5-(trifluoromethyl)benzamide; 3-fluoro-5-(4-(pyrrolidin-1-yl)piperidin-1-yl)-N-(3-(trifluoromethyl)phenyl)benzamide; 3-(4-(pyrrolidin-1-yl)piperidin-1-yl)-N-(3-(trifluoromethyl)phenyl)benzamide; ethyl 2-(3-(4-(pyrrolidin-1-yl)piperidin-1-yl)-5-(trifluoromethyl)benzamido)-4-(trifluoromethyl)thiazole-5-carboxylate; N-(1H-benzo[d]imidazol-2-yl)-3-(4-(pyrrolidin-1-yl)piperidin-1-yl)-5-(trifluoromethyl)benzamide; 3-(3-(dimethylamino)pyrrolidin-1-yl)-5-(trifluoromethyl)-N-(3-(trifluoromethyl)phenyl)benzamide; 3-(4-(pyrrolidin-1-yl)piperidin-1-yl)-5-(trifluoromethyl)-N-(1-(3-(trifluoromethyl)phenyl)cyclopropyl)benzamide; N-(4,5-dicyano-1H-imidazol-2-yl)-3-(4-(pyrrolidin-1-yl)piperidin-1-yl)-5-(trifluoromethyl)benzamide; N-(1H-indol-5-yl)-3-(4-(pyrrolidin-1-yl)piperidin-1-yl)-5-(trifluoromethyl)benzamide; 3-(4-morpholinopiperidin-1-yl)-5-(trifluoromethyl)-N-(3-(trifluoromethyl)phenyl)benzamide; N-(3,5-di-tert-butylphenyl)-3-(4-(pyrrolidin-1-yl)piperidin-1-yl)-5-(trifluoromethyl)benzamide; N-(5-phenylthiazol-2-yl)-3-(4-(pyrrolidin-1-yl)piperidin-1-yl)-5-(trifluoromethyl)benzamide; 3-(3-(piperidin-1-yl)pyrrolidin-1-yl)-5-(trifluoromethyl)-N-(3-(trifluoromethyl)phenyl)benzamide; 3-(4-(pyrrolidin-1-yl)piperidin-1-yl)-5-(trifluoromethyl)-N-(4-(trifluoromethyl)phenyl)benzamide; 3-(4-(pyrrolidin-1-yl)piperidin-1-yl)-N-(4-(trifluoromethoxy)phenyl)-5-(trifluoromethyl)benzamide; N-(3,5-bis(trifluoromethyl)phenyl)-3-(4-(pyrrolidin-1-yl)piperidin-1-yl)-5-(trifluoromethyl)benzamide; N-(4-methyl-2-oxo-1,2-dihydroquinolin-6-yl)-3-(4-(pyrrolidin-1-yl)piperidin-1-yl)-5-(trifluoromethyl)benzamide; N-(4-(4-chlorophenyl)thiazol-2-yl)-3-(4-(pyrrolidin-1-yl)piperidin-1-yl)-5-(trifluoromethyl)benzamide; 3-(4-(4-methylpiperazin-1-yl)piperidin-1-yl)-5-(trifluoromethyl)-N-(3-(trifluoromethyl)phenyl)benzamide; N-(3-chlorophenyl)-3-(4-(4-methylpiperazin-1-yl)piperidin-1-yl)-5-(trifluoromethyl)benzamide; 3-(4,4′-bipiperidin-1-yl)-5-(trifluoromethyl)-N-(3-(trifluoromethyl)phenyl)benzamide; 3-(4-(pyrrolidin-1-yl)piperidin-1-yl)-N-(3-(trifluoromethoxy)phenyl)-5-(trifluoromethyl)benzamide; 3-(3,4-dihydroisoquinolin-2(1H)-yl)-5-(trifluoromethyl)-N-(3-(trifluoromethyl)phenyl)benzamide; 3-(4-(pyrrolidin-1-yl)piperidin-1-yl)-N-(6-(trifluoromethoxy)benzo[d]thiazol-2-yl)-5-(trifluoromethyl)benzamide; 3-(4-(pyrrolidin-1-yl)piperidin-1-yl)-5-(trifluoromethyl)-N-(3-(trifluoromethyl)phenyl)benzamide; 3-(4-methyl-1,4′-bipiperidin-1′-yl)-5-(trifluoromethyl)-N-(3-(trifluoromethyl)phenyl)benzamide; N-(4-phenylthiazol-2-yl)-3-(4-(pyrrolidin-1-yl)piperidin-1-yl)-5-(trifluoromethyl)benzamide; 3-(1,4′-bipiperidin-1′-yl)-5-(trifluoromethyl)-N-(3-(trifluoromethyl)phenyl)benzamide; N-(3-cyanophenyl)-3-(4-(pyrrolidin-1-yl)piperidin-1-yl)-5-(trifluoromethyl)benzamide; N-(3-chlorophenyl)-3-(4-(pyrrolidin-1-yl)piperidin-1-yl)-5-(trifluoromethyl)benzamide; N-(4-(4-bromophenyl)thiazol-2-yl)-3-(4-(pyrrolidin-1-yl)piperidin-1-yl)-5-(trifluoromethyl)benzamide; N-(5-(propylsulfonyl)-1H-benzo[d]imidazol-2-yl)-3-(4-(pyrrolidin-1-yl)piperidin-1-yl)-5-(trifluoromethyl)benzamide; N-(4-chlorophenyl)-3-(4-(pyrrolidin-1-yl)piperidin-1-yl)-5-(trifluoromethyl)benzamide; N-(6-chloropyridin-3-yl)-3-(4-(pyrrolidin-1-yl)piperidin-1-yl)-5-(trifluoromethyl)benzamide; N-(3-fluoro-5-(trifluoromethyl)phenyl)-3-(4-(pyrrolidin-1-yl)piperidin-1-yl)-5-(trifluoromethyl)benzamide; N-(4-bromo-3-chlorophenyl)-3-(4-(pyrrolidin-1-yl)piperidin-1-yl)-5-(trifluoromethyl)benzamide; N-(3-chlorophenyl)-3-(4-methyl-1,4′-bipiperidin-1′-yl)-5-(trifluoromethyl)benzamide; N-(5-chlorothiazol-2-yl)-3-(4-(pyrrolidin-1-yl)piperidin-1-yl)-5-(trifluoromethyl)benzamide; N-(3-methoxy-5-(trifluoromethyl)phenyl)-3-(4-(pyrrolidin-1-yl)piperidin-1-yl)-5-(trifluoromethyl)benzamide; 3-(1,4′-bipiperidin-1′-yl)-N-(3-chlorophenyl)-5-(trifluoromethyl)benzamide; N-(6-chlorobenzo[d]thiazol-2-yl)-3-(4-(pyrrolidin-1-yl)piperidin-1-yl)-5-(trifluoromethyl)benzamide; 3-(3-methyl-1,4′-bipiperidin-1′-yl)-5-(trifluoromethyl)-N-(3-(trifluoromethyl)phenyl)benzamide; N-(4-fluoro-3-(trifluoromethyl)phenyl)-3-(4-(pyrrolidin-1-yl)piperidin-1-yl)-5-(trifluoromethyl)benzamide; N-(3,4-dichlorophenyl)-3-(4-(pyrrolidin-1-yl)piperidin-1-yl)-5-(trifluoromethyl)benzamide; N-(3-(4-methyl-1H-imidazol-1-yl)-5-(trifluoromethyl)phenyl)-3-(4-(pyrrolidin-1-yl)piperidin-1-yl)-5-(trifluoromethyl)benzamide; N-(3,5-dichlorophenyl)-3-(4-(pyrrolidin-1-yl)piperidin-1-yl)-5-(trifluoromethyl)benzamide; N-(biphenyl-4-yl)-3-(4-(pyrrolidin-1-yl)piperidin-1-yl)-5-(trifluoromethyl)benzamide; N-(4-((4-ethylpiperazin-1-yl)methyl)-3-(trifluoromethyl)phenyl)-3-(4-(pyrrolidin-1-yl)piperidin-1-yl)-5-(trifluoromethyl)benzamide; N-(4-bromo-3-(trifluoromethyl)phenyl)-3-(4-(pyrrolidin-1-yl)piperidin-1-yl)-5-(trifluoromethyl)benzamide; 2-(4-(pyrrolidin-1-yl)piperidin-1-yl)-N-(3-(trifluoromethyl)phenyl)isonicotinamide; N-(3-chlorophenyl)-2-(4-(pyrrolidin-1-yl)piperidin-1-yl)isonicotinamide; 3-(4-(pyrrolidin-1-yl)piperidin-1-yl)-5-(trifluoromethyl)-N-(3-(trifluoromethyl)phenyl)aniline; 3-(4-(pyrrolidin-1-yl)piperidin-1-yl)-5-(trifluoromethyl)-N-(3-(trifluoromethyl)benzyl)aniline; 2-(4-(pyrrolidin-1-yl)piperidin-1-yl)-4-(trifluoromethyl)-N-(3-(trifluoromethyl)phenyl)pyrimidine-5-carboxamide; N-(3-chlorophenyl)-2-(4-(pyrrolidin-1-yl)piperidin-1-yl)-4-(trifluoromethyl)pyrimidine-5-carboxamide; N-(3-chlorophenyl)-6-methyl-2-(4-(pyrrolidin-1-yl)piperidin-1-yl)pyrimidine-4-carboxamide; 6-tert-butyl-2-(4-(pyrrolidin-1-yl)piperidin-1-yl)-N-(3-(trifluoromethyl)phenyl)pyrimidine-4-carboxamide; and 6-tert-butyl-N-(3-chlorophenyl)-2-(4-(pyrrolidin-1-yl)piperidin-1-yl)pyrimidine-4-carboxamide.

In a further embodiment are compounds selected from: N-(3-chlorophenyl)-3-(4-methyl-1H-imidazol-1-yl)-5-(trifluoromethyl)benzamide; 3-(4-methyl-1H-imidazol-1-yl)-5-(trifluoromethyl)-N-(3-(trifluoromethyl)phenyl)benzamide; 3-chloro-N-(3-(4-methyl-1H-imidazol-1-yl)-5-(trifluoromethyl)phenyl)benzamide; N-(4-((4-ethylpiperazin-1-yl)methyl)-3-(trifluoromethyl)phenyl)-3-(trifluoromethyl)benzamide; N-(3-chlorophenyl)-3-(1-methylpiperidin-4-yloxy)-5-(trifluoromethyl)benzamide; 3-chloro-N-(4-((4-ethylpiperazin-1-yl)methyl)-3-(trifluoromethyl)phenyl)benzamide; 3-(1-methylpiperidin-4-yloxy)-5-(trifluoromethyl)-N-(3-(trifluoromethyl)phenyl)benzamide; 3-(4-methyl-1H-imidazol-1-yl)-5-(trifluoromethyl)-N-(3-(trifluoromethyl)phenyl)benzamide; N-(3-(trifluoromethyl)-5-(3-(trifluoromethyl)phenylcarbamoyl)phenyl)piperidine-3-carboxamide; 3-(2-(piperidin-1-yl)ethylamino)-5-(trifluoromethyl)-N-(3-(trifluoromethyl)phenyl)benzamide; (S)-3-((1-ethylpyrrolidin-2-yl)methylamino)-5-(trifluoromethyl)-N-(3-(trifluoromethyl)phenyl)-benzamide.

In a further embodiment is a method for treating a Plasmodium related disease in a subject to prevent, inhibit or ameliorate the pathology and/or symptamology of the Plasmodium related disease, comprising administering to a subject, in vivo or in vitro, a therapeutically effective amount of a compound of the invention alone or in combination with a second agent.

In a further embodiment, the Plasmodium related disease is malaria.

In a further embodiment, the second agent is selected from a kinase inhibitor, an anti-malarial drug and an anti-inflammatory agent. The anti-malarial drug is selected from proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, and pyronaridine.

In a further embodiment, the compounds of the invention can be administered prior to, simultaneously with, or after the second agent.

In a further embodiment, the subject is a human.

### Pharmacology and Utility

Compounds of the invention are useful in the treatment and/or prevention of infections such as those caused by Plasmodium falciparum; Plasmodium vivax; Plasmodium ovale; and Plasmodium malaria, trypanosoma cruzi and parasites of the Leishmania genus, such as, for example, Leishmania donovani.

Malaria is an infectious disease caused by four protozoan parasites: Plasmodium falciparum; Plasmodium vivax; Plasmodium ovale; and Plasmodium malaria. These four parasites are typically transmitted by the bite of an infected female Anopheles mosquito. Malaria is a problem in many parts of the world and over the last few decades the malaria burden has steadily increased. An estimated 1-3 million people die every year from malaria—mostly children under the age of 5. This increase in malaria mortality is due in part to the fact that Plasmodium falciparum, the deadliest malaria parasite, has acquired resistance against nearly all available antimalarial drugs, with the exception of the artemisinin derivatives.

Leishmaniasis is caused by one or more than 20 varieties of parasitic protozoa that belong to the genus Leishmania, and is transmitted by the bite of female sand flies. Leishmaniasis is endemic in about 88 countries, including many tropical and sub-tropical areas.

There are four main forms of Leishmaniasis. Visceral leishmaniasis, also called kala-azar, is the most serious form and is caused by the parasite Leishmania donovani. Patients who develop visceral leishmaniasis can die within months unless they receive treatment. The two main therapies for visceral leishmaniasis are the antimony derivatives sodium stibogluconate (Pentostam®) and meglumine antimoniate (Glucantim®). Sodium stibogluconate has been used for about 70 years and resistance to this drug is a growing problem. In addition, the treatment is relatively long and painful, and can cause undesirable side effects.

Human African Trypanosomiasis, also known as sleeping sickness, is a vector-borne parasitic disease. The parasites concerned are protozoa belonging to the Trypanosoma Genus. They are transmitted to humans by tsetse fly (Glossina Genus) bites which have acquired their infection from human beings or from animals harboring the human pathogenic parasites.

Chagas disease (also called American Trypanosomiasis) is another human parsitic disease that is endemic amongst poor populations on the American continent. The disease is caused by the protozoan parasite Trypanosoma cruzi, which is transmitted to humans by blood-sucking insects. The human disease occurs in two stages: the acute stage, which occurs shortly after infection and the chronic stage, which can develop over many years. Chronic infections result in various neurological disorders, including dementia, damage to the heart muscle and sometimes dilation of the digestive tract, as well as weight loss. Untreated, the chronic disease is often fatal.

The drugs currently available for treating Chagas disease are Nifurtimox and benznidazole. However, problems with these current therapies include their diverse side effects, the length of treatment, and the requirement for medical supervision during treatment. Furthermore, treatment is really only effective when given during the acute stage of the disease. Resistance to the two frontline drugs has already occurred. The antifungal agent Amphotericin b has been proposed as a second-line drug, but this drug is costly and relatively toxic.

The phylum, Apicomplexa, contains many members that are human or animal pathogens including, but not limited to, Plasmodium spp. (Malaria), Toxoplasma gondii (congenital neurological defects in humans), Eimeria spp. (poultry and cattle pathogens), Cryptosporidia (opportunistic human and animal pathogens), Babesia (cattle parasites) and Theileria (cattle parasites). The pathogenesis associated with these parasitic diseases is due to repeated cycles of host-cell invasion, intracellular replication and host-cell lysis. Therefore, understanding parasite proliferation is essential for development of novel drugs and vaccines, for example, to treat malaria.

In vertebrate hosts, the parasite undergoes two main phases of development, the hepathocytic and erythrocytic phases, but it is the erythrocytic phase of its life cycle that causes severe pathology. During the erythrocytic phase, the parasite goes through a complex but well synchronized series of stages, suggesting the existence of tightly regulated signaling pathways.

Calcium serves as an intracellular messenger to control synchronization and development in the erythrocytic life phase. The Plasmodium spp. genomes reveal many sequence identities with calcium binding/sensing protein motifs that include Pf39, calmodulin, and calcium dependent protein kinases (CDPKs). Plasmodium CDPKs, Plasmodium CDPK3 and 4, have been shown to be involved in mosquito infection. CDPK4 has been demonstrated to be essential for the sexual reproduction in the midgut of mosquito by translating the calcium signal into a cellular response and regulating cell cycle progression in the male gametocyte. CDPK3 regulates ookinete gliding motility and penetration of the layer covering the midgut epithelium. P. falciparum CDPK1 (PfCDPK1) is expressed during late schizogony of blood stage and in the infectious sporozoite stage and is secreted to the parasitophorous vacuole by an acylation-dependent mechanism. It can be myristoylated and is abundantly found in detergent-resistant membrane fractions isolated from schizogony-phase parasites. Ontology based pattern identification analysis reveals that PfCDPK1 is clustered with genes associated with either parasite egress or erythrocyte invasion. Direct inhibition of PfCDPK1 can arrest the parasite erythrocytic life cycle progression in the late schizogony phase.

Therefore, kinase activity is distributed in all the stages of P. falciparum parasite maturation and kinase inhibitors of the present invention can be used for treating Plasmodium related diseases. In particular, kinase inhibitors of the present invention can be a route for treating malaria by inhibiting the kinase PfCDPK1. The in vitro cellular assay, infra, can be used to assess the activity of compounds of the invention against a variety of malarial parasite strains.

In accordance with the foregoing, the present invention further provides a method for preventing or treating malaria in a subject in need of such treatment, which method comprises administering to said subject a therapeutically effective amount of a compound of Formula I or a pharmaceutically acceptable salt thereof. The required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). Non-limiting examples of compounds which can be used in combination with compounds of the invention are known anti-malarial drugs, for example, proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, pyronaridine, etc.

Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I can be prepared by proceeding as in the following Reaction Scheme I:

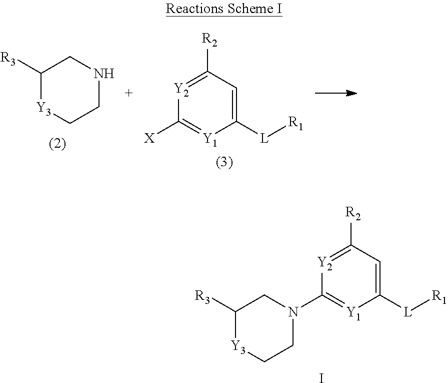

in which R₁, R₂, R₃, Y₁, Y₂, Y₃, m and n are as defined for Formula I in the Summary of the Invention and X is a leaving group (such as halo, sulfones, sulfonates, and the like).

Compounds of Formula I can be prepared by reacting a compound of formula 2 with a compound of formula 3 in the presence of a suitable solvent (for example, dichloromethane, chloroform, dimethylsulfoxide, N,N-dimethyl formamide, butanols, toluene, xylene and the like) using an appropriate base (for example, triethylamine, diisopropyl ethyl amine, sodium carbonate, and the like) and optionally an appropriate metal catalyst (for example, palladium, nickel, gold, copper, and the like). The reaction proceeds at a temperature range of about 5 to about 200° C. and can take up to 24 hours to complete.

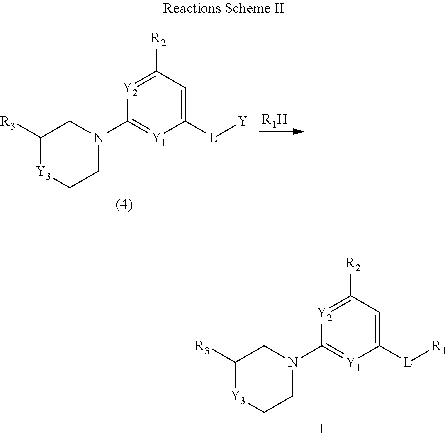

in which R₁, R₂, R₃, Y₁, Y₂, Y₃, m and n are as defined for Formula I in the Summary of the Invention and Y is a protecting group (such as a carbamate, ester, and the like).

Compounds of Formula I can be prepared by reacting a compound of formula 4 with R₁H in the presence of a suitable solvent (for example, dichloromethane, chloroform, dimethylsulfoxide, N,N-dimethyl formamide, butanols, toluene, xylene and the like) using an appropriate base (for example, triethyl amine, diisopropyl ethyl amine, sodium carbonate and the like) and optionally an appropriate activating agent (for example, N,N′-Dicyclohexylcarbodiimide, O-(7-Azabenzotriazol-1-yl)-N,N,N′,N′-tetramethyluronium hexafluorophosphate, mixed anhydrides, thionyl chloride, phorphorus oxychloride, and the like). The reaction proceeds at a temperature range of about 5 to about 50° C. and can take up to 48 hours to complete.

Detailed descriptions of the synthesis of compounds of the Invention are given in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme I & II; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following Examples and intermediates (Reference compounds) that illustrate the preparation of compounds of the invention.

### Reference Compound 1C and 1D

### sodium 3-(4-(pyrrolidin-1-yl)piperidin-1-yl)-5-(trifluoromethyl)benzoate and 3-(4-(pyrrolidin-1-yl)piperidin-1-yl)-5-(trifluoromethyl)aniline

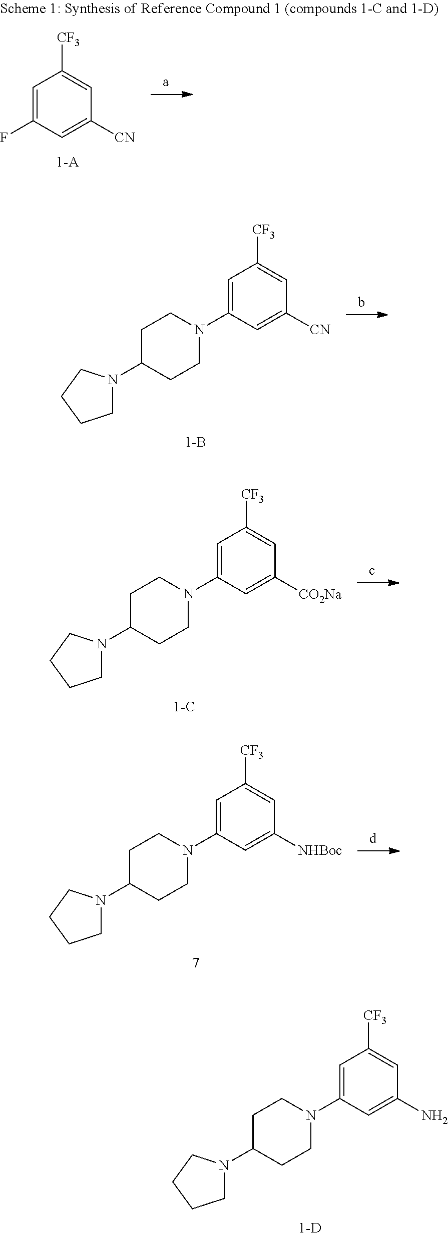

Reagents and conditions: (a) 4-(pyrrolidine-1-yl)piperidine, K₂CO₃, DMSO, 80° C.; (b) i. 50% H₂SO₄, reflux; ii. NaOH, 2 steps; (c) DPPA, Et₃N, DCM, rt; (d). TFA, DCM, rt.

1-B: To a stirred solution of 1-A (6.49 g, 34 2 mmol) in 50 mL of DMSO is added 4-(pyrrolidine-1-yl)piperidine (5.80 g, 37.6 mmol) and K₂CO₃ (5.20 g, 37.6 mmol) at room temperature. The reaction mixture is stirred at 80° C. for 1 hour. HPLC/MS test indicates the complete consumption of 1-A) and a single new peak with the right mass for 1-B ([M+1]=324).

The reaction mixture is diluted with ethyl acetate and washed with water and brine. The organic solution is dried over Na₂SO₄ and concentrated. The crude product is used directly in the next step without further purification.

1-C: 1-B (11.06 g, 34.2 mmol) is added to 90 mL 1:1 concentrated sulfuric acid and water. The reaction mixture is stirred at reflux for 2 hours. HPLC/MS test indicates the complete consumption of 1-B and a single new peak with the right mass for the acid form of 1-C.

The reaction mixture is cooled to room temperature and neutralized carefully by 8 N NaOH solution with ice water cooling. Solvent is completely removed. Ethanol (3×100 mL) is added to the dried residue and boiled for 10 minutes. Solid is filtered off and the combined filtrates are concentrated to give a light yellow solid. ¹H NMR (CD₃OD, 400 MHz) 7.77 (1H, s), 7.66 (1H, s), 7.27 (1H, s), 3.91-3.94 (2H, m), 3.56-3.63 (1H, m), 3.42 (4H, br), 2.83-2.89 (2H, m), 2.21-2.24 (2H, m), 2.07-2.11 (4H, m), 1.82-1.92 (2H, m). MS m/z=343 (M+1).

7: To a solution of 1-C (3.0 g, 8.23 mmol) in 30 mL of anhydrous t-BuOH is added DPPA (2.14 mL, 9.88 mmol) and Et₃N (1.49 mL, 9.88 mmol). The reaction mixture is stirred at reflux for 10 hours under nitrogen atmosphere. HPLC/MS test indicates the complete consumption of 1-C and a major peak with the right mass for 7 ([M+1]=414).

The reaction mixture is cooled to room temperature. Solvent is removed. The residue is subjected to flash column chromatography purification (12 g, 0-10% MeOH in DCM) to give a yellow oil.

Intermediate 7 is dissolved in 10 mL of 20% TFA in DCM. The reaction mixture is stirred for 2 hours at room temperature. HPLC/MS test shows a single major peak with the right mass for 1-D. Solvent is removed. The residue is dissolved in DCM and is extracted twice with 30 mL of 1N HCl. The combined aqueous layers are washed with DCM and neutralized carefully with 4N NaOH. The resulting aqueous solution is extracted with 100 mL of DCM for 3 times. The combined DCM solution is dried over Na₂SO₄ and concentrated to give 850 mg (33%) yellow oil. H NMR (CDCl₃, 400 MHz) 6.54 (1H, s), 6.35 (1H, s), 6.33 (1H, t, J=2.0 Hz), 3.3.63-3.68 (2H, m), 2.72-2.79 (2H, m), 2.59-2.62 (4H, br), 2.11-2.18 (1H, m), 1.96-1.99 (2H, m), 1.78-1.82 (4H, m), 1.59-1.69 (2H, m).MS m/z=314 (M+1).

### Reference Compound 2

### sodium 3-(4-(pyrrolidin-1-yl)piperidin-1-yl)benzoate

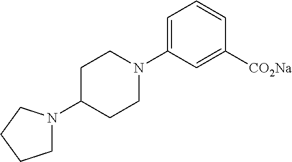

This compound is prepared starting from 3-fluorobenzonitrile using method analogous to those described for the preparation of Reference Compound 1. MS m/z 275.2 (M+1).

### Reference Compound 3

### 3-fluoro-5-(4-(pyrrolidin-1-yl)piperidin-1-yl)aniline

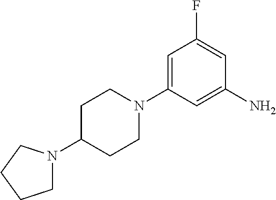

This compound is prepared starting from 1,3-difluoro-5-nitrobenzene using method analogous to those described for the preparation of Reference Compound 1-B. To a solution of nitro precursor (1.47 g, 5.0 mmol) in 25 mL of ethanol is added 10% Pd/C (106 mg, 0.1 mmol). The reaction mixture is degassed and back-filled with H₂ and stirred at room temperature under H₂ overnight. HPLC/MS test indicates presence of product ([M+1]=264). Material is filtered and solvent is evaporated to give a yellow oil which is used without further purification. ¹H NMR (CD₃OD, 400 MHz) 6.14 (1H, t, J=2.0 Hz), 6.04 (1H, dt, J=2.0, 12.4 Hz), 5.93 (1H, dt, J=2.0, 10.8 Hz), 3.69-3.72 (2H, m), 2.67-2.79 (6H, m), 2.31-2.39 (1H, m), 2.03-2.07 (2H, m), 1.86-1.89 (4H, m), 1.58-1.68 (2H, m). MS m/z 264.2 (M+1).

### Reference Compound 4

### 3-(1-methylpiperidin-4-yloxy)-5-(trifluoromethyl)benzoic acid

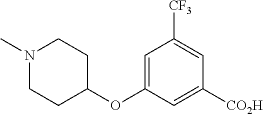

This compound is prepared starting from 3-fluoro-5-(trifluoromethyl)-benzonitrile and 1-methylpiperidin-4-ol using a method analogous to those described for the preparation of Reference Compound 1. MS m/z 304.2 (M+1).

### Reference Compound 5

### 3-(4-methyl-1H-imidazol-1-yl)-5-(trifluoromethyl)benzoic acid

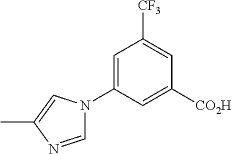

This compound is prepared starting from 3-fluoro-5-(trifluoromethyl)benzonitrile and 4-methyl-1H-imidazole using a method analogous to that described for the preparation of Reference Compound 1. MS m/z=312.4 (M+1).

### Reference Compound 6

### 3-(4-methyl-1H-imidazol-1-yl)-5-(trifluoromethyl)aniline

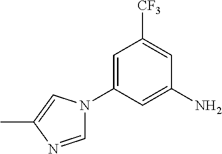

This compound is prepared using method analogous to those described for the preparation of reference compound 1-E from 1-C. MS m/z=283.45 (M+1).

### Reference Compound 7

### 4-((4-ethylpiperazin-1-yl)methyl)-3-(trifluoromethyl)aniline

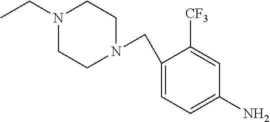

To a solution of 1-methyl-4-nitro-2-trifluoromethyl-benzene (15 g, 73.1 mmol, 1.0 eq.) in carbon tetrachloride (250 ml) is added NBS (13 g, 73.1 mmol, 1.0 eq.) and AIBN (1.19 g, 7.31 mmol, 0.1 eq.) as an initiator. The reaction mixture is refluxed overnight and then partitioned with water. The organic layer is separated and the aqueous layer is extracted with dichloromethane. The combined organic extracts are washed with water, dried over Na₂SO₄, filtered and concentrated to afford the solids. The solids are dissolved in dichloromethane (300 ml). The clear solution is treated with DIEA (12.55 ml, 73.1 mmol, 1.0 eq.) and N-Ethylpiperazine (8.25 g, 73.1 mmol, 1.0 eq.). The reaction mixture is stirred at room temperature for 30 min (till the completion of reaction as per LCMS). The reaction mixture is washed with water, dried over Na₂SO₄, filtered and concentrated to afford the crude product, which is purified by flash column chromatography (Hexanes/ethyl acetate=1/1) to afford 1-Ethyl-4-(4-nitro-2-trifluoromethyl-benzyl)-piperazine (11.57 g, 50%) as a solid.

To a solution 4-(4-ethyl-piperazin-1-ylmethyl)-3-trifluoromethyl-phenylamine (10 g, 31.54 mmol, 1.0 eq.) in MeOH (250 ml) is added Raney Nickel (1.0 g, 10 wt %). The suspension is stirred under hydrogen atmosphere (1 atm) for 24 hours. The reaction mixture is then filtered over celite and the filtrate is concentrated under reduced pressure to yield the desired product: MS m/z=421.26 (M+1).

### Reference Compound 8

### sodium 3-(1,4′-bipiperidin-1′-yl)-5-(trifluoromethyl)benzoate

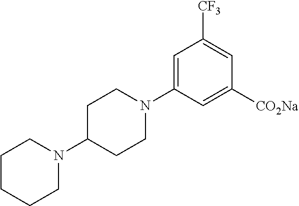

This compound is prepared starting from 3-fluoro-5-(trifluoromethyl)benzonitrile and 1,4′-bipiperidine using method analogous to those described for the preparation of Reference Compound 1. ¹H NMR (CD₃OD, 400 MHz) 7.82 (1H, s), 7.69 (1H, s), 7.43 (1H, s), 4.02-4.05 (2H, m), 3.55-3.58 (2H, br), 3.37-3.44 (1H, m), 3.01-3.07 (2H, m), 2.90-2.96 (2H, m), 2.22 (2H, d, J=12.4 Hz), 2.00 (2H, d, J=12.4 Hz), 1.78-1.90 (6H, m). MS m/z 357.2 (M+1).

### Reference Compound 9

### sodium 3-(4-methyl-1,4′-bipiperidin-1′-yl)-5-(trifluoromethyl)benzoate

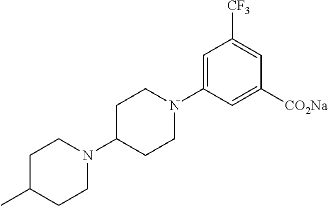

This compound is prepared starting from 3-fluoro-5-(trifluoromethyl)benzonitrile and 4-methyl-1,4′-bipiperidine using method analogous to those described for the preparation of Reference Compound 1. MS m/z 371.2 (M+1).

### Reference Compound 10

### sodium 3-(piperidin-1-yl)-5-(trifluoromethyl)benzoate

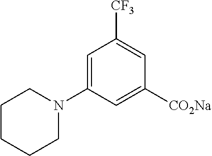

This compound is prepared starting from 3-fluoro-5-(trifluoromethyl)benzonitrile and piperidine using method analogous to those described for the preparation of Reference Compound 1. MS m/z 274.2 (M+1).

### Reference Compound 11

### sodium 2-(4-(pyrrolidin-1-yl)piperidin-1-yl)isonicotinate

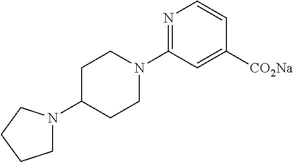

This compound is prepared from 2-chloroisonicotinonitrile and 4-(pyrrolidine-1-yl)piperidine using method analogous to those described for the preparation of Reference compound 1. ¹H NMR (CD₃OD, 400 MHz) 8.22 (1H, d, J=5.6 Hz), 7.60 (1H, s), 7.28 (1H, d, J=5.6 Hz), 4.50-4.53 (2H, m), 3.71 (2H, br), 3.47-3.53 (1H, m), 3.10-3.21 (4H, m), 2.30-2.33 (2H, m), 2.19 (2H, br), 2.04 (2H, br), 1.74-1.84 (2H, m). MS m/z 276.2 (M+1).

### Reference Compound 12

### 6-tert-butyl-2-(4-(pyrrolidin-1-yl)piperidin-1-yl)pyrimidine-4-carboxylic acid

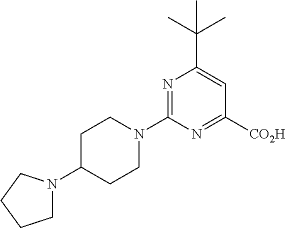

Synthesis of Reference Compound 12. Reagents and conditions: (a) urea, HCl, EtOH, reflux; (b) POCl₃, DIEA, MeCN, reflux; (c) 4-(pyrrolidine-1-yl)piperidine, DIEA, MeCN, 120° C.; (d) LiOH, MeOH/H₂O (3:1), 60° C.:

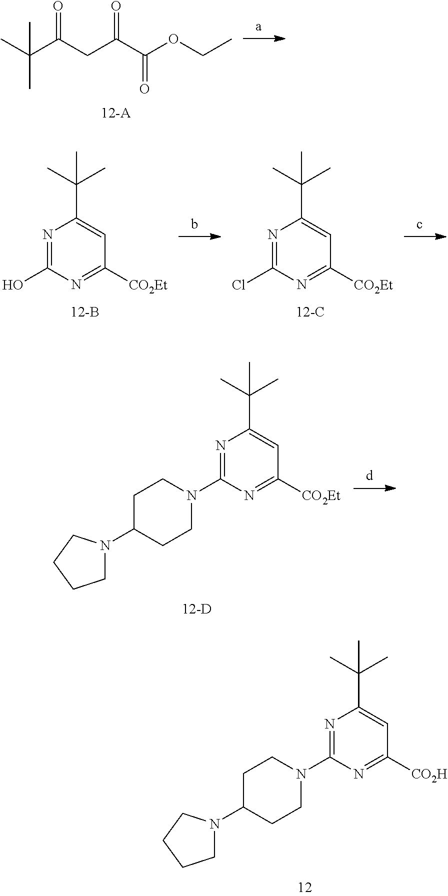

2-B: A solution of 12-A (2.0 g, 10 mmol), urea (961 mg, 16 mmol) and 1 mL of concentrated HCl in 80 mL of ethanol is stirred at reflux for 2 days. LCMS indicated that starting material is consumed and the desired product is produced as the major product. Solvent is removed and the residue is directly subjected to flash column chromatography separation (40 g, 10-90% ethyl acetate in hexane) to give a white solid.

2-C: To a solution of 12-B (1.01 g, 4.5 mmol) in 10 mL of MeCN is added POCl₃ (2.1 mL, 22.5 mmol) and DIEA (784 μL, 4.5 mmol). The reaction mixture is stirred at reflux for 2 hours under nitrogen. HPLC/MS test showed that 12-B is disappeared and desired product 12-C ([M+1]=243) is the major product. The reaction mixture is cooled to room temperature. Solvent is removed and the residue dissolved in ethyl acetate and washed with NaHCO₃ and brine. The organic solution is dried and concentrated to give a yellow oil as crude product.

12-D: A solution of 12-C (485 mg, 2.0 mmol), 4-(pyrrolidine-1-yl)piperidine (309 mg, 2.0 mmol) and DIEA (1.74 mL, 10.0 mmol) in 10 mL of MeCN is stirred 120° C. with oil bath for 2 days. HPLC-MS test showed that 12-C is consumed and 12-D is the major product. The reaction mixture is cooled to room temperature. The solvent is removed. The product is used in the next step without further purification.

12: To a solution of 12-D (541 mg, 1.5 mmol) in 15 mL of methanol and 5 mL of water is added LiOH (180 mg, 7.5 mmol). The reaction mixture is stirred at 60° C. for 3 hours. HPLC/MS test showed that 12-D is disappeared and desired product 12 ([M+1]=333) is the major product. The reaction mixture is cooled to room temperature and solvent is removed. The reaction mixture is dissolved in methanol and subjected to MS-triggered HPLC separation to give a yellow oil. MS m/z 333.3 (M+1).

### Reference Compound 13

### sodium 6-methyl-2-(4-(pyrrolidin-1-yl)piperidin-1-yl)pyrimidine-4-carboxylate

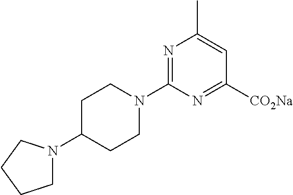

This compound is prepared from methyl 2-chloro-6-methylpyrimidine-4-carboxylate using method analogous to those described for the preparation of intermediate 12 from 12C. ¹H NMR (CD₃OD, 400 MHz) 7.10 (1H, s), 5.04-5.08 (2H, m), 3.66 (2H, br), 3.40-3.48 (1H, m), 3.19 (2H, br), 2.91-2.98 (2H, m), 2.42 (3H, s), 2.20-2.23 (2H, m), 2.10 (4H, br), 1.58-1.68 (2H, m). MS m/z 291.2 (M+1)

### Reference Compound 14

### sodium 2-(4-(pyrrolidin-1-yl)piperidin-1-yl)-6-(trifluoromethyl)pyrimidine-4-carboxylate

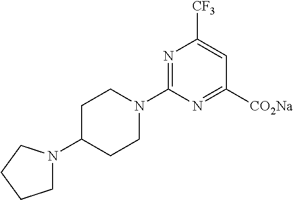

This compound is prepared from methyl 2-chloro-6-methylpyrimidine-4-carboxylate using method analogous to those described for the preparation of intermediate 12. from 12C. MS m/z 345.2 (M+1).

### Reference Compound 15

### sodium 3-(4-(4-methylpiperazin-1-yl)piperidin-1-yl)-5-(trifluoromethyl)benzoate

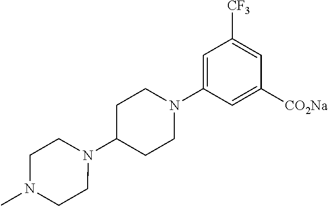

This compound is prepared starting from 3-fluoro-5-(trifluoromethyl)benzonitrile and 1-methyl-4-(piperidin-4-yl)piperazine using method analogous to those described for the preparation of Reference Compound 1. ¹H NMR (CD₃OD, 400 MHz) 7.79 (1H, s), 7.67 (1H, s), 7.23 (1H, s), 3.86-3.89 (2H, m), 2.80-2.97 (10H, m), 2.64-2.67 (1H, m), 2.63 (3H, s), 2.00-2.03 (2H, d, J=12.0 Hz), 1.62-1.72 (2H, m). MS m/z 373.2 (M+1).

### Reference Compound 16

### sodium 3-fluoro-5-(4-(pyrrolidin-1-yl)piperidin-1-yl)benzoate

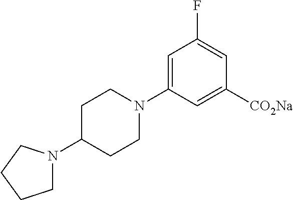

This compound is prepared starting from 3,5-difluorobenzonitrile and 4-(pyrrolidine-1-yl)piperidine using method analogous to those described for the preparation of Reference Compound 1. ¹H NMR (CD₃OD, 400 MHz) 7.43 (1H, s), 7.13 (1H, d, J=8.8 Hz), 6.97 (1H, d, J=12.0 Hz), 3.93-3.96 (2H, m), 3.67 (2H, br), 3.34-3.38 (1H, m), 3.19 (2H, br), 2.85-2.92 (2H, m), 2.23-2.26 (2H, m), 2.15 (2H, br), 2.04 (2H, br), 1.74-1.84 (2H, m). MS m/z 293.2 (M+1).

### Reference Compound 17

### 3-bromo-5-(trifluoromethyl)-N-(3-(trifluoromethyl)phenyl)benzamide

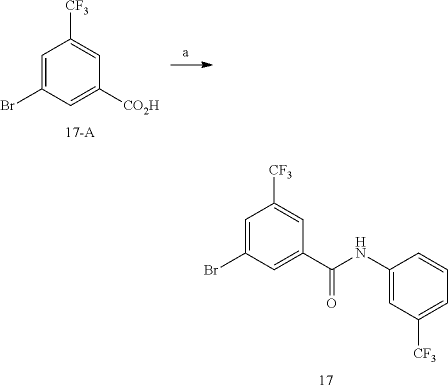

To a stirred solution of 17-A (2.69 g, 10 mmol) in 50 mL of DMF are added HATU (3.80 g, 10 mmol) and DIEA (5.23 mL, 30 mmol). After stirring for 10 minutes, 3-(trifluoromethyl)aniline (1.88 mL, 15 mmol) is added. The reaction mixture is stirred at room temperature over the weekend. HPLC/MS test showed that desired product 17 is the major product. The reaction mixture is diluted with ethyl acetate, washed with NH₄Cl and brine. The organic solution is dried and concentrated. The residue is subjected to flash column chromatography purification (120 g, 10-30% ethyl acetate in hexane) to give 3.02 g (73%) white solid. MS m/z 412.0 (M+1).

### Reference Compound 18

### 3-morpholino-5-(trifluoromethyl)benzoic acid

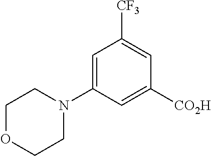

This compound is prepared starting from 3-fluoro-5-(trifluoromethyl)benzonitrile and morpholine using method analogous to those described for the preparation of Reference Compound 1. MS m/z 276.1 (M+1).

### Example 1

To a stirred solution of Ref. Comp. 1-D (10 mg, 0.031 mmol) in 0.3 mL of dichloromethane added DIEA (′2 μL, 1.10 eq.), and methanesulfonyl chloride (7.2 μL , 1.0 eq.). The reaction mixture is stirred at room temperature for 3 hours. HPLC/MS test showed that the starting material (amine) is gone and desired product 1 is the major peak. Solvent is removed under reduced pressure. The residue is directly taken to mass triggered HPLC separation. The collected MeCN/water solution is concentrated and dried to give Example 1.

### Example 2

This compound is prepared from Reference Compound 1-D and 3-(trifluoromethyl)benzene-1-sulfonyl chloride using method analogous to that described for the preparation of Example 1.

### Example 3

This compound is prepared from Reference Compound 1-D and 3-acetamidobenzene-1-sulfonyl chloride using method analogous to those described for the preparation of Example 1.

### Example 4

This compound is prepared from Reference Compound 1-C and 4-methylbenzene-1-sulfonyl chloride using method analogous to those described for the preparation of Example 1.

### Example 5

This compound is prepared from Reference Compound 1-D and 4-tert-butylbenzene-1-sulfonyl chloride using method analogous to those described for the preparation of Example 1.

### Example 6

This compound is prepared from Reference Compound 1-D and 4-methoxybenzene-1-sulfonyl chloride using method analogous to those described for the preparation of Example 1.

### Example 7

This compound is obtained as an intermediate during the synthesis of Reference Compound 1-D.

### Example 8

To a stirred solution of Ref. Comp. 1-D (10 mg, 0.031 mmol) in 0.3 mL of dichloromethane added DIEA (6 μL, 1.10 eq.), and cyclopropanecarbonyl chloride (2.8 μL , 1.0 eq.). The reaction mixture is stirred at room temperature for 3 hours. HPLC/MS test showed that the starting material (amine) is gone and desired product 8 is the major peak. Solvent is removed under reduced pressure. The residue is directly taken to mass triggered HPLC separation. The collected MeCN/water solution is concentrated and dried on lyophilizer to give powdery product.

### Example 9

This compound is prepared from Reference Compound 1-D and 2-chloronicotinoyl chloride using method analogous to those described for the preparation of Example 8.

### Example 10

This compound is prepared from Reference Compound 1-D and 2-Fluorobenzoyl chloride using method analogous to those described for the preparation of Example 9.

### Example 11

This compound is prepared from Reference Compound 3 and 3-chlorobenzoic acid using method analogous to those described for the preparation of Example 82.

### Example 12

This compound is prepared from Reference Compound 3 and 3-trifluoromethylbenzoic acid using method analogous to those described for the preparation of Example 82.

### Example 13

This compound is prepared from Reference Compound 1-D and 2,4-Dichlorobenzoyl chloride using method analogous to those described for the preparation of Example 9.

### Example 14

This compound is prepared from Reference Compound 1-D and 4-methoxybenzoic anhydride using method analogous to those described for the preparation of Example 1.

### Example 15

This compound is prepared from Reference Compound 1-D and 3-Cyano-benzoylchloride using method analogous to those described for the preparation of Example 8.

### Example 16

This compound is prepared from Reference Compound 1-D and 3-methylbenzoyl chloride using method analogous to those described for the preparation of Example 8.

### Example 17

This compound is prepared from Reference Compound 1-D and 3-fluorobenzoyl chloride using method analogous to those described for the preparation of Example 8

### Example 18

This compound is prepared from Reference Compound 1-D and 3-trifluorobenzoyl chloride using method analogous to those described for the preparation of Example 8.

### Example 19

This compound is prepared from Reference Compound 1-C and 1-D using method analogous to those described for the preparation of Example 82.

### Example 20

To a stirred solution of Reference. Compound. 1-D (20 mg, 0.062 mmol) in 0.6 mL of DMF added DIEA (12 μL, 1.10 eq.), and 4-Chloro-2-methyl phenylisocyanate (10 gm, 1.0 eq.). The reaction mixture is stirred at 50° C. for 8 hours. HPLC/MS test showed that the starting material (amine) is gone and desired product 20 is the major peak. Solvent is removed under reduced pressure. The residue is directly taken to mass triggered HPLC separation. The collected MeCN/water solution is concentrated and dried on lyophilizer to give powdery product.

### Example 21

This compound is prepared from Reference Compound 1-D and 3-Chlorophenyl isocyanate using method analogous to those described for the preparation of Example 20.

### Example 22

To a solution of 3-(trifluoromethyl)aniline (0.02 gm, 1.0 eq.), in dichloromethane (1.0 ml) are added 4-nitrophenyl chloroformate (0.027 gm, 1.05 eq.) and pyridine (0.01 gm, 1.05 eq.). The reaction mixture is stirred for 5 minutes, after which Reference compound 1-D (0.04 g, 1.0 eq.) and N,N-diisopropyl ethylamine (0.017 ml, 1.0.5 eq.) are added. The resulting reaction is stirred for 1 hour at ambient temperature after which time LC-MS analysis revealed disappearance of 1-D. The solvent is removed in vacuo and the resulting residue is dissolved in DMSO (1 ml). The resulting solution is purified by reverse-phase LC-MS to yield the title compound as the trifluoroacetic acid salt.

### Example 23

This compound is prepared from Reference Compound 1-D and 3-(4-methyl-1H-imidazol-1-yl)-5-(trifluoromethyl)aniline using method analogous to those described for the preparation of Example 22.

### Example 24

This compound is prepared from Reference Compound 1-D and 3,5-dichloroaniline using method analogous to those described for the preparation of Example 22.

### Example 25

This compound is prepared from Reference Compound 1-C and 1-D using method analogous to those described for the preparation of Example 22.

### Example 26

This compound is prepared from Reference Compound 9 and methyl 2-(4-aminophenyl)-2-methylpropanoate using method analogous to those described for the preparation of Example 82. The hydrolysis of methyl ester is carried out analogous to synthesis of Reference Compound 12.

### Example 27

This compound is prepared from Reference Compound 1-C and 4-(trifluoromethyl)thiazol-2-amine using method analogous to those described for the preparation of Example 82.

### Example 28

This compound is prepared from Reference Compound 18 and 3-Chlorobenzoic acid using method analogous to those described for the preparation of Example 82.

### Example 29

This compound is prepared from Reference Compound 17 and piperidine-3-carboxamide using method analogous to those described for the preparation of Example 81.

### Example 30

This compound is prepared from Reference Compound 1-C and 4-amino-N-(thiazol-2-yl)benzenesulfonamide using method analogous to those described for the preparation of Example 82.

### Example 31

This compound is prepared from Reference Compound 1-C and butylamine using method analogous to those described for the preparation of Example 82.

### Example 32

This compound is prepared from Reference Compound 1-C and 6-(trifluoromethyl)pyrimidin-4-amine using method analogous to those described for the preparation of Example 82.

### Example 33

To a stirred solution of Reference Compound 1-C (72 mg, 0.20 mmol) in 5 mL of chloroform is added 1 mL of SOCl₂. After stirring at reflux for 1 hour, solvent is removed. 5 mL of pyridine is added and followed by the addition of N-Me-3-Chloroaniline (36 μL, 0.30 mmol). The reaction mixture is stirred at room temperature overnight. Formation of product is assessed by HPLC/MS. Solvent is removed. The residue is separated by mass triggered HPLC, concentrated and dried to give product.

### Example 34

This compound is prepared from Reference Compound 1-C and 2-chloropyridin-4-amine using method analogous to those described for the preparation of Example 82.

### Example 35

This compound is prepared from Reference Compound 18 and 3-Trifluoromethylbenzoic acid using method analogous to those described for the preparation of Example 82.

### Example 36

This compound is prepared from Reference Compound 1-C and 3-aminobenzamide using method analogous to those described for the preparation of Example 82.

### Example 37

This compound is prepared from Reference Compound 1-C and 4-(2-chlorophenyl)thiazol-2-amine using method analogous to those described for the preparation of Example 82.

### Example 38

This compound is prepared from Reference Compound 1-C and 3-chlorophenyl hydrazine using method analogous to those described for the preparation of Example 82.

### Example 39

This compound is prepared from Reference Compound 1-C and 3-Trifluoromethylphenyl hydrazine using method analogous to those described for the preparation of Example 82.

### Example 40

This compound is prepared from Reference Compound 10 and 3-chloroaniline using method analogous to those described for the preparation of Example 82.

### Example 41

This compound is prepared from Reference Compound 1-C and 4-aminopyrimidine using method analogous to those described for the preparation of Example 82.

### Example 42

This compound is prepared from Reference Compound 1-C and 6-(trifluoromethyl)indoline using method analogous to those described for the preparation of Example 82.

### Example 43

This compound is prepared from Reference Compound 1-C and 2-chloroaniline using method analogous to those described for the preparation of Example 82.

### Example 44

This compound is prepared from Reference Compound 2 and 3-chloroaniline using method analogous to those described for the preparation of Example 82.

### Example 45

This compound is prepared from Reference Compound 17 and 1-(3-(trifluoromethyl)phenyl)piperazine using method analogous to those described for the preparation of Example 81.

### Example 46

This compound is prepared from Reference Compound 10 and 3-Trifluoromethyl aniline using method analogous to those described for the preparation of Example 82.

### Example 47

This compound is prepared from Reference Compound 17 and 1,4′-bipiperidin-2-one using method analogous to those described for the preparation of Example 81.

### Example 48

This compound is prepared from Reference Compound 1-C and 4-tert-butylthiazol-2-amine using method analogous to those described for the preparation of Example 82.

### Example 49

This compound is prepared from Reference Compound 17 and tert-butyl piperidin-4-ylcarbamate using method analogous to those described for the preparation of Example 81.

### Example 50

This compound is prepared from Reference Compound 1-C and 1-(3-(trifluoromethyl)phenyl)piperazine using method analogous to those described for the preparation of Example 82.

### Example 51

This compound is prepared from Reference Compound 1-C and 3,4-Dimethylthiazol-2-amine using method analogous to those described for the preparation of Example 82.

### Example 52

This compound is prepared from Reference Compound 1-C and 3-Chloro-4-methoxyaniline using method analogous to those described for the preparation of Example 82.

### Example 53

This compound is prepared from Reference Compound 17 and 1-(piperidin-4-yl)pyrrolidin-2-one using method analogous to those described for the preparation of Example 81.

### Example 54

This compound is prepared from Reference Compound 1-C and 4-Morpholinoaniline using method analogous to those described for the preparation of Example 82.

### Example 55

This compound is prepared from Reference Compound 1-C and 3-Trifluoromethyl benzylamine using method analogous to those described for the preparation of Example 82.

### Example 56

This compound is prepared from Reference Compound 1-C and 3-(1H-pyrazol-4-yl)aniline using method analogous to those described for the preparation of Example 82.

### Example 57

This compound is prepared from Reference Compound 17 and Ethyl piperidine-3-carboxylate using method analogous to those described for the preparation of Example 81.

### Example 58

This compound is prepared from Reference Compound 1-C and 1H-indazol-6-amine using method analogous to those described for the preparation of Example 82.

### Example 59

This compound is prepared from Reference Compound 1-C and aniline using method analogous to those described for the preparation of Example 82.

### Example 60

This compound is prepared from Reference Compound 1-C and ethyl 2-amino-4-(trifluoromethyl)thiazole-5-carboxylate using method analogous to those described for the preparation of Example 82.

### Example 61

This compound is prepared from Reference Compound 16 and 3-Trifluoromethyl aniline using method analogous to those described for the preparation of Example 82.

### Example 62

This compound is prepared from Reference Compound 2 and 3-Trifluoromethyl aniline using method analogous to those described for the preparation of Example 82.

### Example 63

This compound is prepared from Reference Compound 17 and N,N-dimethylpyrrolidin-3-amine using method analogous to those described for the preparation of Example 81.

### Example 64

This compound is prepared from Reference Compound 1-C and 1H-benzo[d]imidazol-2-amine using method analogous to those described for the preparation of Example 82.

### Example 65

This compound is prepared from Reference Compound 1-C and 1-(3-(trifluoromethyl)phenyl)cyclopropanamine using method analogous to those described for the preparation of Example 82.

### Example 66

This compound is prepared from Reference Compound 1-C and 3,4-Dicyano-2-aminoimidazole using method analogous to those described for the preparation of Example 82.

### Example 67

This compound is prepared from Reference Compound 1-C and 5-Aminoindole using method analogous to those described for the preparation of Example 82.

### Example 68

This compound is prepared from Reference Compound 17 and 4-(piperidin-4-yl)morpholine using method analogous to those described for the preparation of Example 81.

### Example 69

This compound is prepared from Reference Compound 1-C and 3,5-tert-butylaniline using method analogous to those described for the preparation of Example 82.

### Example 70

This compound is prepared from Reference Compound 1-C and 5-phenylthiazol-2-amine using method analogous to those described for the preparation of Example 82.

### Example 71

This compound is prepared from Reference Compound 17 and 1-(pyrrolidin-3-yl)piperidine using method analogous to those described for the preparation of Example 81.

### Example 72

This compound is prepared from Reference Compound 1-C and 4-Trifluoromethyl aniline using method analogous to those described for the preparation of Example 82.

### Example 73

This compound is prepared from Reference Compound 1-C and 4-Trifluoromethoxy aniline using method analogous to those described for the preparation of Example 82.

### Example 74

This compound is prepared from Reference Compound 1-C and 3,5-Ditrifluoromethyl aniline using method analogous to those described for the preparation of Example 82.

### Example 75

This compound is prepared from Reference Compound 1-C and 6-Amino-4-methylquinolin-2(1H)-one using method analogous to those described for the preparation of Example 82.

### Example 76

This compound is prepared from Reference Compound 1-C and 4-(4-chlorophenyl)thiazol-2-amine using method analogous to those described for the preparation of Example 82.

### Example 77

This compound is prepared from Reference Compound 15 and 3-Trifluoromethylaniline using method analogous to those described for the preparation of Example 82.

### Example 78

This compound is prepared from Reference Compound 15 and 3-Chloroaniline using method analogous to those described for the preparation of Example 82.

### Example 79

This compound is prepared from Reference Compound 17 and 4,4′-bipiperidine using method analogous to those described for the preparation of Example 81.

### Example 80

This compound is prepared from Reference Compound 1-C and 3-Trifluoromethoxy aniline using method analogous to those described for the preparation of Example 82.

### Example 81

### 3-(3,4-dihydroisoquinolin-2(1H)-yl)-5-(trifluoromethyl)-N-(3-(trifluoromethyl)-phenyl)benzamide

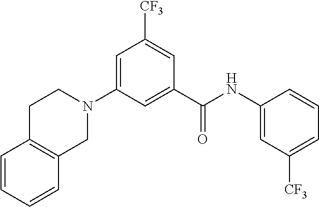

Reagents and conditions: (a) 1,2,3,4-tetrahydroisoquinoline, Pd₂(dba)₃, BINAP, tBuOK, toluene, 100° C. To a stirred solution of Reference Compound 17 (41 mg, 0.10 mmol) in 3 mL of toluene is added 1,2,3,4-tetrahydroisoquinoline (24 μL, 0.20 mmol), Pd₂(dba)₃ (4.6 mg, 0.005 mmol), BINAP (9.3 mg, 0.015 mmol) and ^{t}BuOK (34 mg, 0.30 mmol). The reaction mixture is degassed and back filled with N₂. The reaction mixture is stirred at 100° C. for 3 hours. HPLC/MS test showed that the starting material (bromide) is gone and desired product 5-C is the major peak. The reaction mixture is diluted with ethyl acetate. Solid is filtered off. The filtrate is washed with brine and concentrated. The residue is subjected to MS-triggered HPLC separation. The collected MeCN/water solution is concentrated and dried to give the product as a TFA salt.

### Example 82

### 3-(4-(pyrrolidin-1-yl)piperidin-1-yl)-5-(trifluoromethyl)-N-(3-(trifluoromethyl)phenyl)benzamide

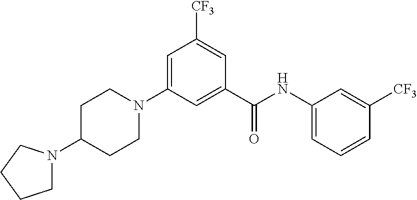

Reagents and conditions: (a) 3-trifluoromethylaniline, HATU, DIEA, DMF, rt. 3-A: To a stirred solution of 1-C (36 mg, 0.10 mmol) in 1 mL of DMF are added HATU (59 mg, 0.15 mmol) and DIEA (52 μL, 0.30 mmol). After 10 minutes' stirring, 3-trifluoromethylaniline (19 μL, 0.15 mmol) is added. The reaction mixture is stirred at room temperature overnight. HPLC/MS test showed that 1-C is all consumed and desired product 82 is the major product.

The reaction mixture is directly taken to mass-triggered HPLC purification. The combined elutes is concentrated till no more MeCN is left. NaHCO₃ is added to the aqueous solution and extracted with DCM. The solution is dried and concentrated to give yellow oil.

### Example 83

This compound is prepared from Reference Compound 1-C and 6-(trifluoromethoxy)benzo[d]thiazol-2-amine using method analogous to those described for the preparation of Example 82.

### Example 84

This compound is prepared from Reference Compound 9 and 3-Trifluoromethyl aniline using method analogous to those described for the preparation of Example 82.

### Example 85

This compound is prepared from Reference Compound 1-C and 4-phenylthiazol-2-amine using method analogous to those described for the preparation of Example 82.

### Example 86

This compound is prepared from Reference Compound 8 and 3-Trifluoromethyl aniline using method analogous to those described for the preparation of Example 82.

### Example 87

This compound is prepared from Reference Compound 1-C and 3-Cyanoaniline using method analogous to those described for the preparation of Example 82.

### Example 88

This compound is prepared from Reference Compound 1-C and 3-Chloroaniline using method analogous to those described for the preparation of Example 82.

### Example 89

This compound is prepared from Reference Compound 1-C and 4-(4-bromophenyl)thiazol-2-amine using method analogous to those described for the preparation of Example 82.

### Example 90

This compound is prepared from Reference Compound 1-C and 5-(propylsulfonyl)-1H-benzo[d]imidazol-2-amine using method analogous to those described for the preparation of Example 82.

### Example 91

This compound is prepared from Reference Compound 1-C and 4-Chloroaniline using method analogous to those described for the preparation of Example 82.

### Example 92

This compound is prepared from Reference Compound 1-C and 2-chloropyridin-5-amine using method analogous to those described for the preparation of Example 82.

### Example 93

This compound is prepared from Reference Compound 1-C and 3-fluoro-5-(trifluoromethyl)aniline using method analogous to those described for the preparation of Example 82.

### Example 94

This compound is prepared from Reference Compound 9 and 3-Chloroaniline using method analogous to those described for the preparation of Example 82.

### Example 95

This compound is prepared from Reference Compound 1-C and 4-Bromo-3-chloroaniline using method analogous to those described for the preparation of Example 82.

### Example 96

This compound is prepared from Reference Compound 1-C and 3-Methoxy-5-trifluoromethylaniline using method analogous to those described for the preparation of Example 82.

### Example 97

This compound is prepared from Reference Compound 1-C and 5-chlorothiazol-2-amine using method analogous to those described for the preparation of Example 82.

### Example 98

This compound is prepared from Reference Compound 8 and 3-Chloroaniline using method analogous to those described for the preparation of Example 82.

### Example 99

This compound is prepared from Reference Compound 1-C and 5,6-chlorobenzo[d]thiazol-2-amine using method analogous to those described for the preparation of Example 82.

### Example 100

This compound is prepared from Reference Compound 1-C and -3-Fluoro-4-trifluoromethylaniline using method analogous to those described for the preparation of Example 82.

### Example 101

This compound is prepared from Reference Compound 17 and 3-methyl-1,4′-bipiperidine using method analogous to those described for the preparation of Example 81.

### Example 102

This compound is prepared from Reference Compound 1-C and 3,4-Dichloroaniline using method analogous to those described for the preparation of Example 82.

### Example 103

This compound is prepared from Reference Compound 1-C and 3,5-Dichloroaniline using method analogous to those described for the preparation of Example 82.

### Example 104

This compound is prepared from Reference Compound 1-C and Biphenyl-4-amine using method analogous to those described for the preparation of Example 82.

### Example 105

This compound is prepared from Reference Compound 1-C and -3-Bromo-4-trifluoromethylaniline using method analogous to those described for the preparation of Example 82.

### Example 106

This compound is prepared from Reference Compound 1-C and Reference Compound 6 using method analogous to those described for the preparation of Example 82.

### Example 107

This compound is prepared from Reference Compound 1-C and Reference Compound 7 using method analogous to those described for the preparation of Example 82.

### Example 108

This compound is prepared from Reference Compound 5 and 3-Chloroaniline using method analogous to those described for the preparation of Example 82.

### Example 109

This compound is prepared from Reference Compound 6 and 3-Trifluoromethylbenzoic acid using method analogous to those described for the preparation of Example 82.

### Example 110

This compound is prepared from Reference Compound 6 and 3-chlorobenzoic acid using method analogous to those described for the preparation of Example 82.

### Example 111

This compound is prepared from Reference Compound 7 and 3-Trifluoromethylbenzoic acid using method analogous to those described for the preparation of Example 82.

### Example 112

This compound is prepared from Reference Compound 7 and 3-chlorobenzoic acid using method analogous to those described for the preparation of Example 82.

### Example 113

This compound is prepared from Reference Compound 11 and 3-Trifluoromethylaniline using method analogous to those described for the preparation of Example 82.

### Example 114

This compound is prepared from Reference Compound 11 and 3-Chloroaniline using method analogous to those described for the preparation of Example 82.

### Example 115

To a stirred solution of Reference Compound 1-D (70 mg, 0.20 mmol) in 5 mL of DCM are added 3-(trifluoromethyl)benzaldehyde (35 mg, 0.20 mmol) and 0.5 mL of AcOH. The reaction mixture is stirred at room temperature for 1 hour. NaBH(OAc)₃ (85 mg, 0.40 mmol) is added and stirred at room temperature overnight. HPLC test indicated the complete consumption of 1-D and 3-(trifluoromethyl)benzaldehyde. The desired product 115 is the major peak. Reaction mixture is diluted with ethyl acetate and washed with brine. The organic solution is dried and concentrated. The residue is taken to mass-triggered preparative HPLC purification. The residue is dissolved in DCM, washed with NaHCO₃ and brine. The DCM solution is dried and concentrated to give yellow solid.

### Example 116

To a stirred solution of Reference Compound 1-D (175 mg, 0.50 mmol) in 20 mL of dioxane are added 1-iodo-3-(trifluoromethyl)benzene (136 mg, 0.50 mmol), Pd₂(dba)₃ (46 mg, 0.05 mmol), xantphos (87 mg, 0.15 mmol) and Cs₂CO₃ (815 mg, 2.5 mmol). Air is removed and N₂ is back filled. The reaction mixture is stirred at 80° C. overnight. LC/MS test indicated that the desired product 116 is formed. Solid is filtered and the organic solution is concentrated. The residue is taken to mass-triggered preparative HPLC purification. The collected MeCN/water solution is concentrated to give yellow oil.

### Example 117

This compound is prepared from Reference Compound 14 and 3-Trifluoromethylaniline using method analogous to those described for the preparation of Example 82.

### Example 118

This compound is prepared from Reference Compound 14 and 3-Chloroaniline using method analogous to those described for the preparation of Example 82.

### Example 119

This compound is prepared from Reference Compound 13 and 3-Chloroaniline using method analogous to those described for the preparation of Example 82.

### Example 120

This compound is prepared from Reference Compound 4 and 3-Chloroaniline using method analogous to those described for the preparation of Example 82.

### Example 121

This compound is prepared from Reference Compound 4 and 3-Trifluoromethylaniline using method analogous to those described for the preparation of Example 82.

### Example 122

This compound is prepared from Reference Compound 5 and 3-Trifluoromethylaniline using method analogous to those described for the preparation of Example 82.

### Example 123

To a stirred solution of 1-C (36 mg, 0.10 mmol) in 1 mL of DMF are added HATU (57 mg, 0.15 mmol) and DIEA (52 μL, 0.30 mmol). After 10 minutes' stirring at room temperature, 3-Chlorophenol (16 μL, 0.15 mmol) is added. The reaction mixture is stirred at room temperature overnight for 2 hours. HPLC/MS test showed that (I) is gone and 123 is the major product ([M+1]=453).

The reaction mixture is directly subjected to mass-triggered preparative HPLC purification. The collected MeCN/water solution is concentrated. The residue is dissolved in DCM, washed with NaHCO₃ and brine. The DCM solution is dried and concentrated to give yellow solid.

### Example 124

This compound is prepared from Reference Compound 17 and piperidine-3-carboxamide using method analogous to those described for the preparation of Example 81.

### Example 125

This compound is prepared from Reference Compound 17 and 2-(piperidin-1-yl)ethanamine using method analogous to those described for the preparation of Example 81.

### Example 126

This compound is prepared from Reference Compound 17 and (S)-(1-ethylpyrrolidin-2-yl)methanamine using method analogous to those described for the preparation of Example 81.

### Example 127

This compound is prepared from Reference Compound 12 and 3-Trifluoromethylaniline using method analogous to those described for the preparation of Example 82.

### Example 128

This compound is prepared from Reference Compound 12 and 3-chloroaniline using method analogous to those described for the preparation of Example 82.

By repeating the procedures described in the above examples, using appropriate starting materials, the following compounds of Formula I, as identified in Table 1, are obtained. Table 1 also documents the physical data obtained from the associated examples above.

TABLE 1

| | | Physical Data |
|---|---|---|
| Com- | | MS (m/z) and |
| pound | Structure | ¹H NMR |
| 1 | 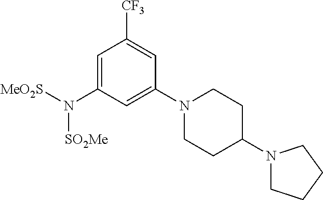 | MS m/z 470.2 (M + 1) |
| 2 | 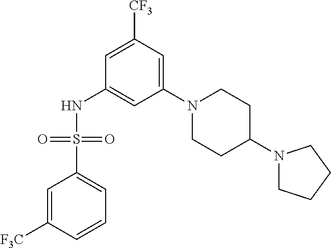 | MS m/z 521.9 (M + 1) |
| 3 | 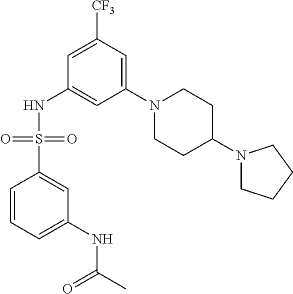 | MS m/z 511.0 (M + 1) |
| 4 | 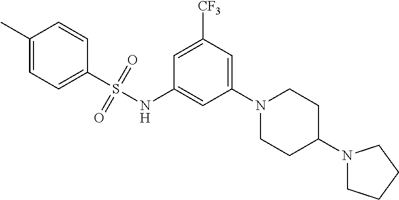 | MS m/z 468.2 (M + 1) |
| 5 | 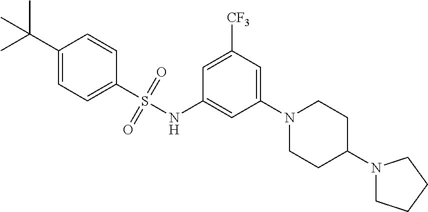 | MS m/z 510.1 (M + 1) |
| 6 | 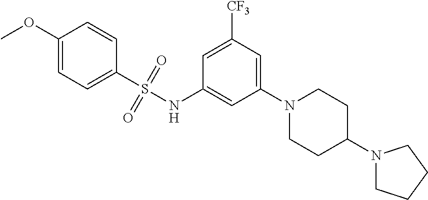 | MS m/z 484.2 (M + 1) |
| 7 | 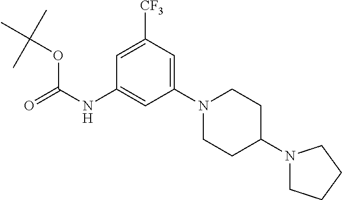 | MS m/z 414.2 (M + 1) ¹H NMR (CD₃OD, 400 MHz) 7.39 (1H, s), 7.15 (1H, s), 6.86 (1H, s), 3.89-3.92 (2H, m), 3.66-3.71 (2H, m), 3.33-3.35 (2H, m), 3.13- 3.25 (2H, m), 2.84-2.91 (1H, m), 2.15-2.26 (4H, m), 1.99-2.07 (2H, m), 1.74-1.84 (2H, m), 1.53 (9H, s). |
| 8 | 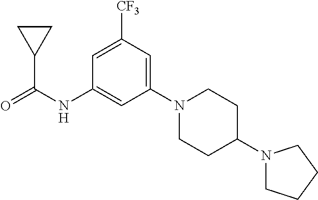 | MS m/z 382.2 (M + 1) |
| 9 | 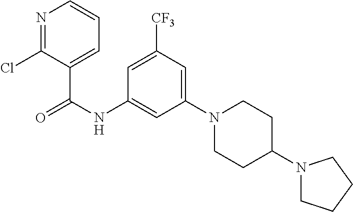 | MS m/z 453.2 (M + 1) |
| 10 | 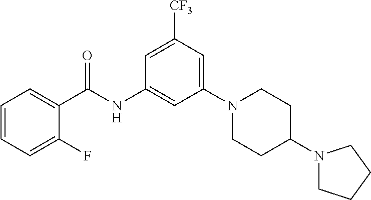 | MS m/z 436.2 (M + 1) |
| 11 | 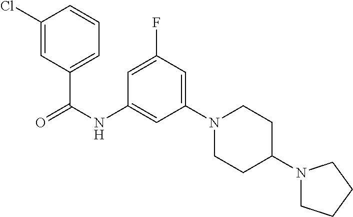 | MS m/z 402.2 (M + 1) ¹H NMR (CDCl₃, 400 MHz) 11.65 (1H, br), 8.43 (1H, s), 7.81-7.82 (1H, m), 7.52-7.54 (1H, m), 7.27-7.31 (1H, m), 7.18 (1H, s), 7.12-7.15 (1H, m), 6.98-7.01 (1H, m), 6.73- 6.77 (1H, s), 3.85-3.88 (4H, m), 3.16-3.18 (1H, m), 2.69-2.94 (4H, m), 2.10-2.20 (8H, m). |
| 12 | 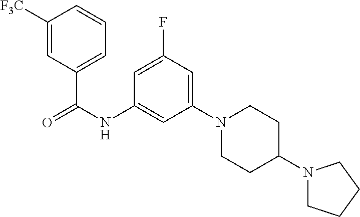 | MS m/z 436.2 (M + 1) |
| 13 | 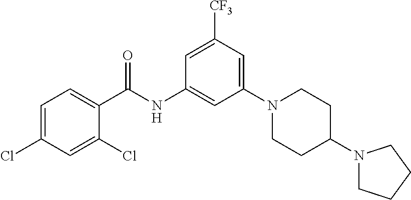 | MS m/z 486.1 (M + 1) |
| 14 | 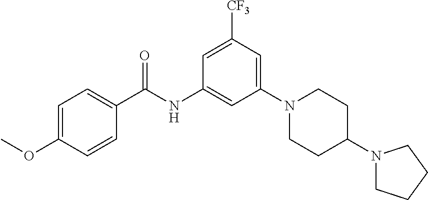 | MS m/z 448.3 (M + 1) ¹H NMR (CD₃OD, 400 MHz) 8.01 (2H, d, J = 8.8 Hz), 7.81 (1H, s), 7.53 (1H, s), 7.12 (2H, d, J = 8.8 Hz), 7.07 (1H, s), 4.01-4.04 (2H, m), 3.96 (3H, s), 3.74-3.78 (2H, m), 3.40-3.46 (1H, m), 3.23-3.29 (2H, m), 2.96-3.02 (2H, m), 2.32-2.35 (2H, m), 2.22-2.28 (2H, m), 2.08-2.14 (2H, m), 1.83-1.93 (2H, m). |
| 15 | 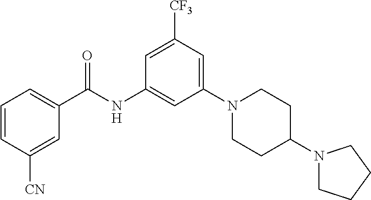 | MS m/z 443.0 (M + 1) |
| 16 | 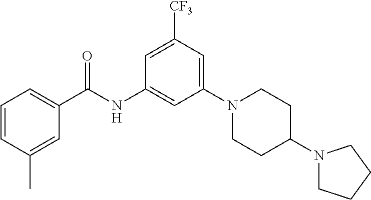 | MS m/z 432.3 (M + 1) |
| 17 | 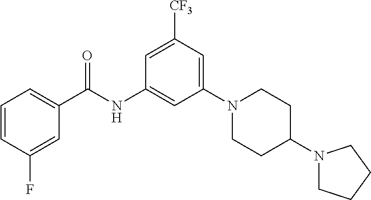 | MS m/z 436.3 (M + 1) |
| 18 | 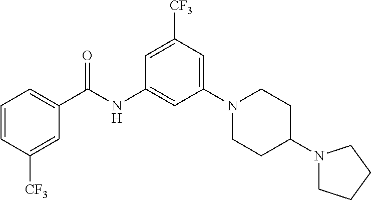 | MS m/z 486.2 (M + 1) |
| 19 | 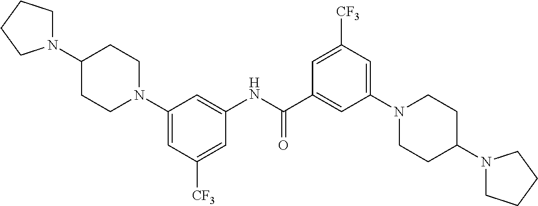 | MS m/z 638.2 (M + 1) |
| 20 | 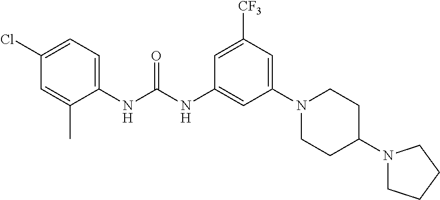 | MS m/z 481.2 (M + 1) |
| 21 | 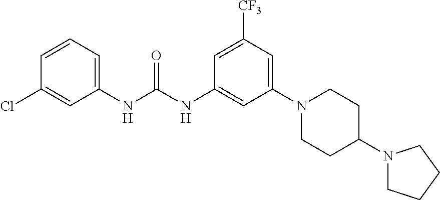 | MS m/z 466.9 (M + 1) |
| 22 | 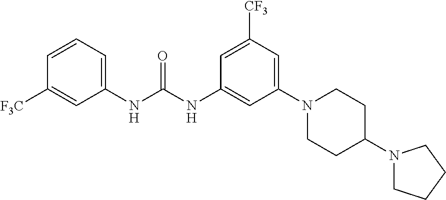 | MS m/z 501.1 (M + 1) |
| 23 | 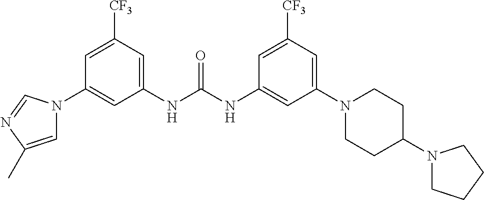 | MS m/z 581.3 (M + 1) |
| 24 | 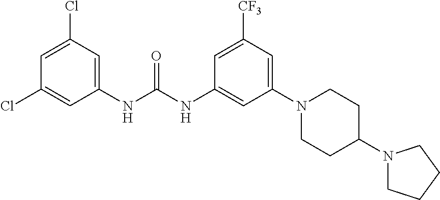 | MS m/z 500.9 (M + 1) |
| 25 | 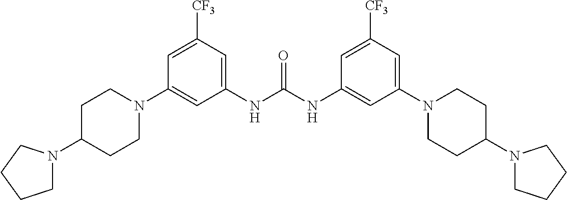 | MS m/z 653.2 (M + 1) |
| 26 | 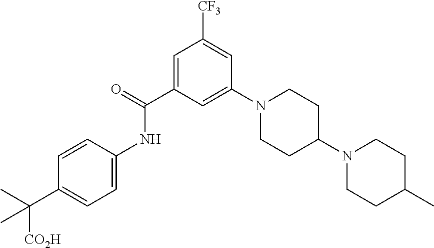 | MS m/z 532.4 (M + 1) |
| 27 | 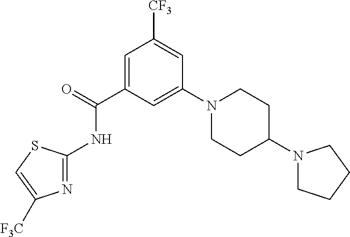 | MS m/z 493.2 (M + 1) |
| 28 | 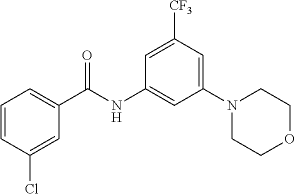 | MS m/z 385.1 (M + 1) |
| 29 | 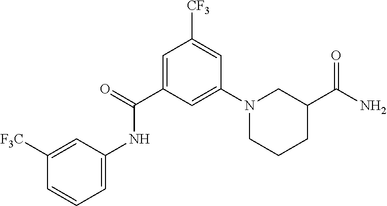 | MS m/z 460.9 (M + 1) |
| 30 | 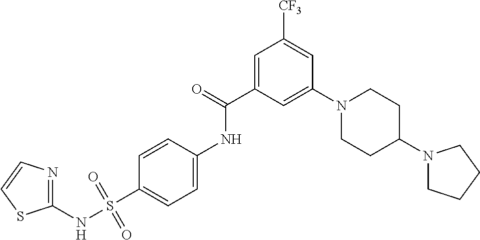 | MS m/z 580.2 (M + 1) |
| 31 | 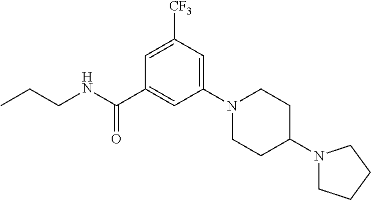 | MS m/z 384.2 (M + 1) ¹H NMR (CD₃OD, 400 MHz) 7.65 (1H, s), 7.54 (1H, s), 7.7.36 (1H, s), 3.99-4.03 (2H, m), 3.68-3.70 (2H, m), 3.34-3.36 (2H, m), 3.32-3.33 (1H, m), 3.15-3.22 (2H, m), 2.90- 2.96 (2H, m), 2.26-2.29 (2H, m), 2.18-2.20 (2H, m), 2.01-2.06 (2H, m), 1.76-1.86 (2H, m), 1.60-1.69 (2H, m), 0.98 (3H, d, J = 7.6 Hz). |
| 32 | 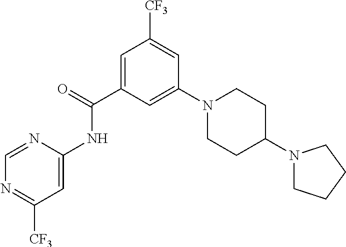 | MS m/z 488.2 (M + 1) |
| 33 | 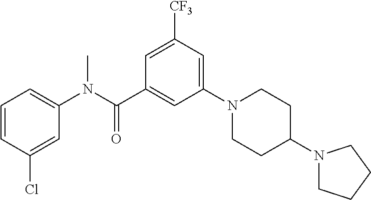 | MS m/z 466.0 (M + 1) |
| 34 | 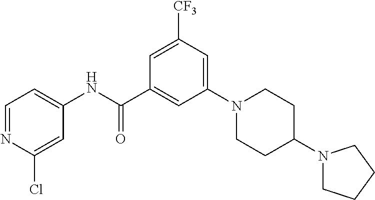 | MS m/z 453.1 (M + 1) |
| 35 | 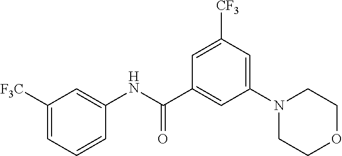 | MS m/z 418.9 (M + 1) |
| 36 | 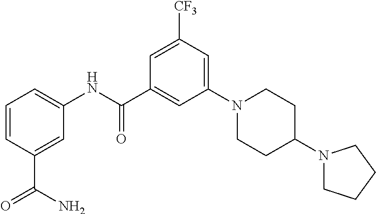 | MS m/z 461.2 (M + 1) |
| 37 | 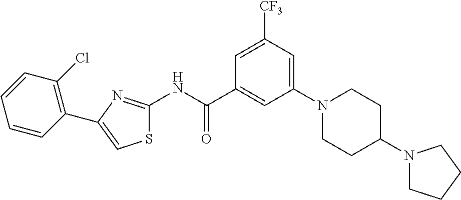 | MS m/z 535.2 (M + 1) |
| 38 | 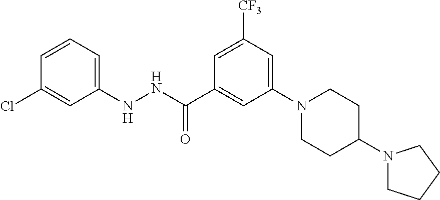 | MS m/z 467.2 (M + 1) |
| 39 | 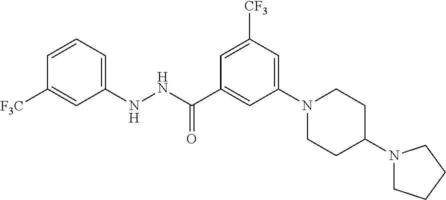 | MS m/z 501.2 (M + 1) ¹H NMR (CD₃OD, 400 MHz) 7.73 (1H, s), 7.64 (1H, s), 7.45 (1H, s), 7.38 (1H, t, J = 8.0 Hz), 7.10 (1H, s), 7.09 (2H, d, J = 7.2 Hz), 4.04-4.07 (2H, m), 3.69 (2H, br), 3.34-3.41 (1H, m), 3.20 (2H, br), 2.93-3.00 (2H, m), 2.27-2.30 (2H, m), 2.19 (2H, br), 2.02 (2H, br), 1.76-1.86 (2H, m). |
| 40 | 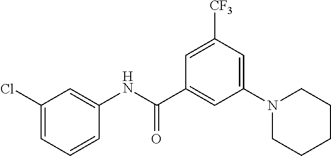 | MS m/z 383.1 (M + 1) |
| 41 | 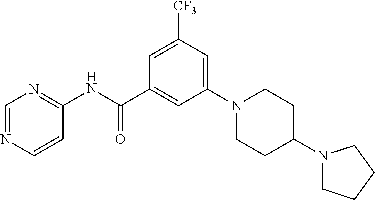 | MS m/z 420.2 (M + 1) |
| 42 | 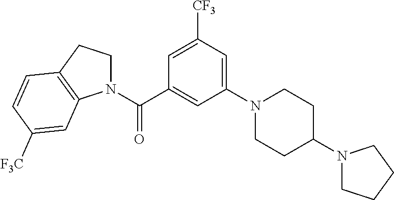 | MS m/z 512.3 (M + 1) |
| 43 | 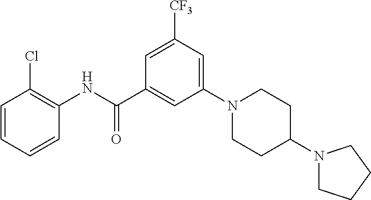 | MS m/z 452.3 (M + 1) |
| 44 | 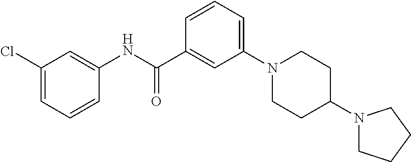 | MS m/z 384.0 (M + 1) |
| 45 | 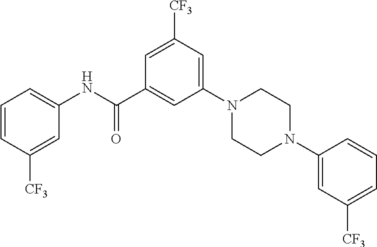 | MS m/z 562.3 (M + 1) ¹H NMR (CD₃OD, 400 MHz) 8.18 (1H, s), 7.98 (1H, d, J = 8.4 Hz), 7.81 (1H, s), 7.69 (1H, s), 7.57 (1H, d, J = 8.0 Hz), 7.42-7.46 (3H, m), 7.27 (1H, d, J = 8.8 Hz), 7.26 (1H, s), 7.12 (1H, d, J = 7.6 Hz), 3.52-3.55 (2H, m), 3.43-3.46 (2H, m). |
| 46 | 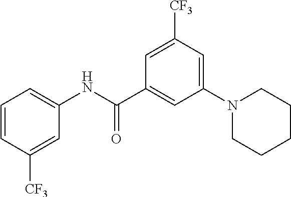 | MS m/z 417.1 (M + 1) |
| 47 | 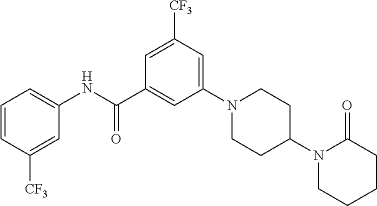 | MS m/z 514.2 (M + 1) |
| 48 | 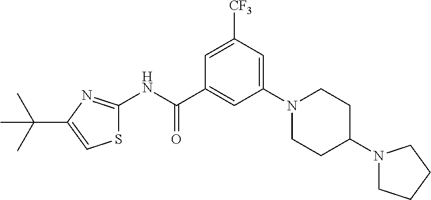 | MS m/z 481.2 (M + 1) |
| 49 | 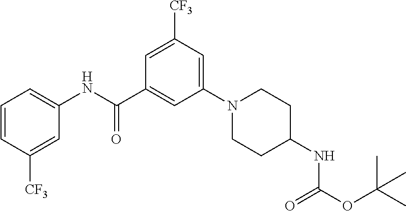 | MS m/z 532.8 (M + 1) |
| 50 | 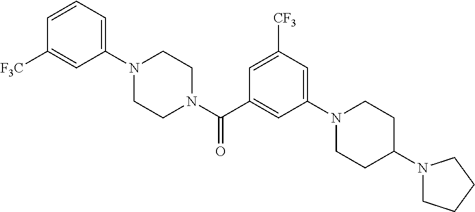 | MS m/z 555.1 (M + 1) |
| 51 | 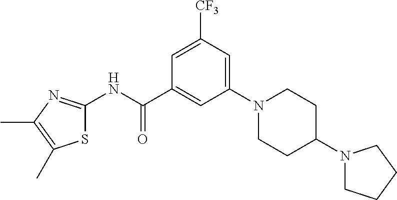 | MS m/z 453.2 (M + 1) |
| 52 | 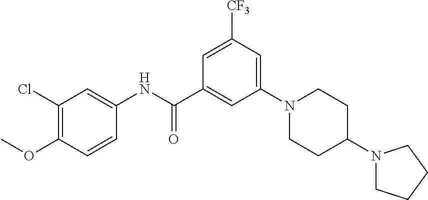 | MS m/z 482.3 (M + 1) |
| 53 | 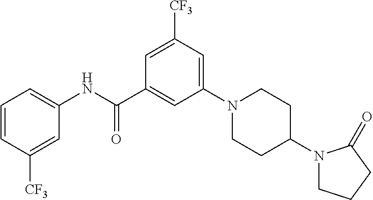 | MS m/z 500.2 (M + 1) ¹H NMR (CD₃OD, 400 MHz) 8.15 (1H, s), 7.60 (1H, d, J = 8.4 Hz), 7.75 (1H, s), 7.64 (1H, s), 7.56 (1H, t, J = 8.0 Hz), 7.44 (1H, d, J = 7.6 Hz), 7.38 (1H, s), 4.06-4.14 (1H, m), 3.98-4.02 (2H, m), 3.46 (2H, t, J = 7.2 Hz), 2.94-3.01 (2H, m), 2.41 (2H, t, J = 7.2 Hz), 2.01-2.08 (2H, m), 1.80-1.95 (4H, m). |
| 54 | 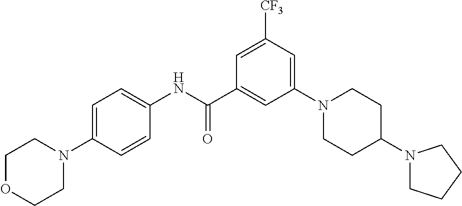 | MS m/z 503.4 (M + 1) |
| 55 | 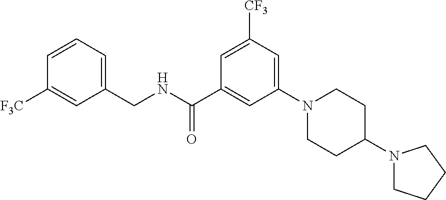 | MS m/z 500.0 (M + 1) |
| 56 | 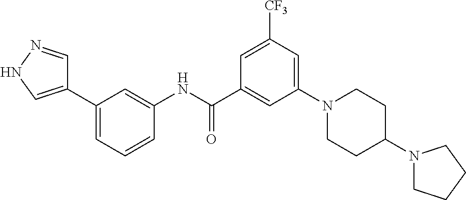 | MS m/z 484.2 (M + 1) |
| 57 | 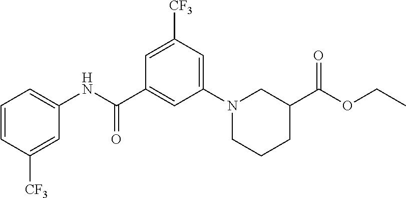 | MS m/z 489.8 (M + 1) |
| 58 | 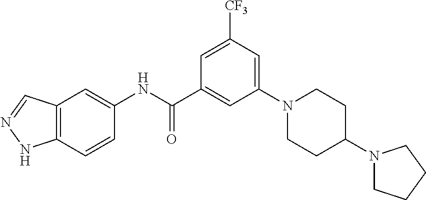 | MS m/z 458.1 (M + 1) |
| 59 | 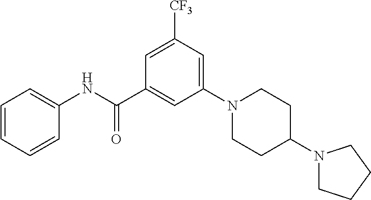 | MS m/z 418.1 (M + 1) ¹H NMR (CD₃OD, 400 MHz) 7.76 (1H, s), 7.71 (1H, s), 7.69 (2H, s), 7.43 (1H, s), 7.38 (2H, t, J = 8.0 Hz), 7.18 (1H, t, J = 8.0 Hz), 4.05-4.09 (2H, m), 3.69 (2H, br), 3.35-3.42 (1H, m), 3.20 (2H, br), 2.94-3.00 (2H, m), 2.28-2.31 (2H, m), 2.19 (2H, br), 2.03 (2H, br), 1.76-1.87 (2H, m). |
| 60 | 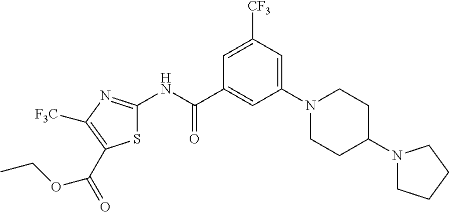 | MS m/z 565.2 (M + 1) |
| 61 | 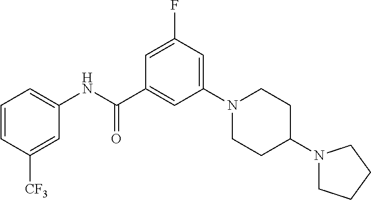 | MS m/z 436.2 (M + 1) |
| 62 | 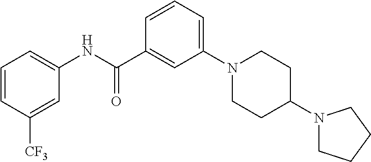 | MS m/z 418.2 (M + 1) |
| 63 | 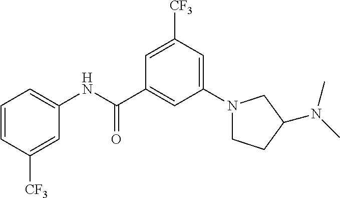 | MS m/z 445.9 (M + 1) |
| 64 | 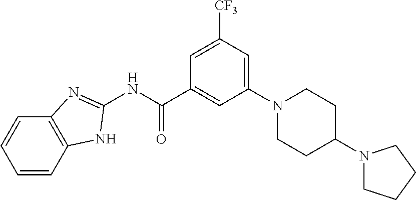 | MS m/z 458.3 (M + 1) |
| 65 | 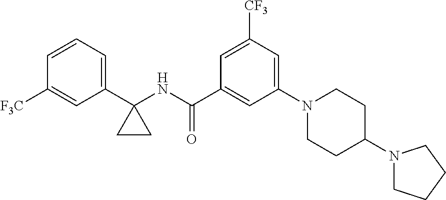 | MS m/z 526.3 (M + 1) |
| 66 | 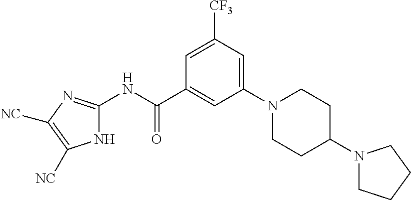 | MS m/z 458.2 (M + 1) |
| 67 | 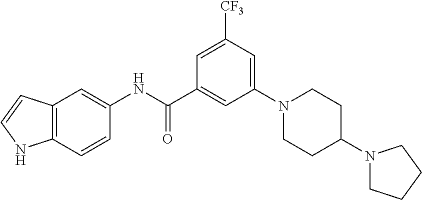 | MS m/z 457.3 (M + 1) ¹H NMR (CD₃OD, 400 MHz) 7.86 (1H, d, J = 2.0 Hz), 7.78 (1H, s), 7.70 (1H, s), 7.41 (1H, s), 7.40 (1H, d, J = 8.8 Hz), 7.32 (1H, dd, J = 2.0, 8.8 Hz), 6.46 (1H, d, J = 2.8 Hz), 4.05-4.08 (2H, m), 3.69 (2H, br), 3.35-3.42 (1H, m), 3.17-3.21 (2H, m), 2.94-3.01 (2H, m), 2.27-2.30 (2H, m), 2.20-2.23 (2H, m), 2.02-2.08 (2H, m), 1.78-1.88 (2H, m). |
| 68 | 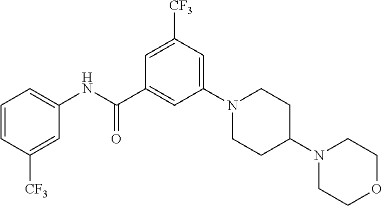 | MS m/z 502.9 (M + 1) ¹H NMR (CD₃OD, 400 MHz) 8.16 (1H, s), 7.95 (1H, d, J = 8.4 Hz), 7.78 (1H, s), 7.71 (1H, s), 7.57 (1H, t, J = 8.0 Hz), 7.46 (1H, d, J = 8.0 Hz), 7.43 (1H, s), 4.10-4.14 (2H, m), 3.95 (4H, br), 3.41-3.49 (5H, m), 2.95-3.02 (2H, m), 2.28-2.31 (2H, m), 1.80- 1.90 (2H, m). |
| 69 | 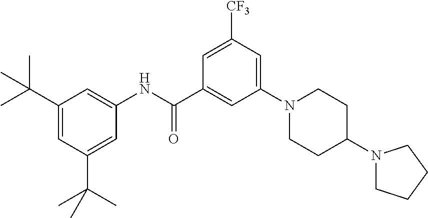 | MS m/z 530.4 (M + 1) |
| 70 | 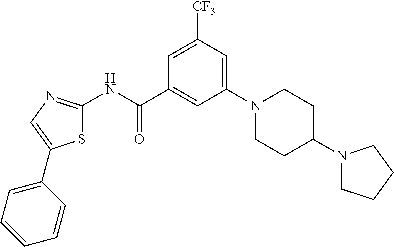 | MS m/z 501.2 (M + 1) |
| 71 | 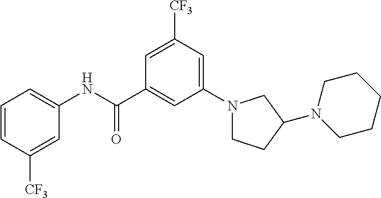 | MS m/z 486.4 (M + 1) |
| 72 | 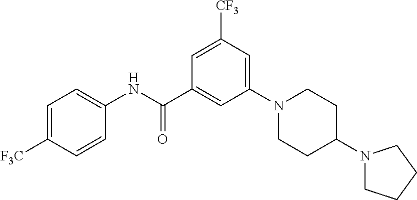 | MS m/z 486.2 (M + 1) |
| 73 | 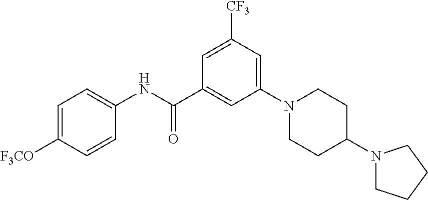 | MS m/z 502.3 (M + 1) ¹H NMR (CD₃OD, 400 MHz) 7.81 (2H, d, J = 8.8 Hz), 7.76 (1H, s), 7.69 (1H, s), 7.42 (1H, s), 7.29 (2H, d, J = 8.0 Hz), 4.05-4.08 (2H, m), 3.67 (2H, br), 3.35-3.41 (1H, m), 3.21 (2H, br), 2.94-3.01 (2H, m), 2.28-2.31 (2H, m), 2.17 (2H, br), 2.11 (2H, br), 1.77- 1.88 (2H, m). |
| 74 | 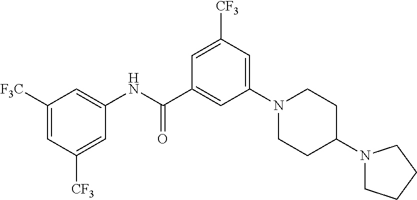 | MS m/z 554.3 (M + 1) |
| 75 | 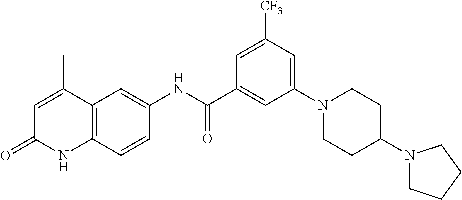 | MS m/z 499.1 (M + 1) |
| 76 | 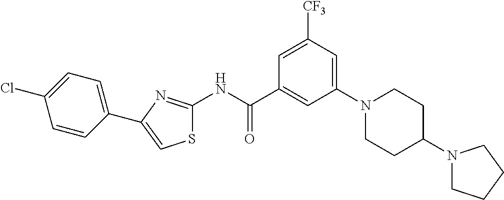 | MS m/z 535.1 (M + 1) |
| 77 | 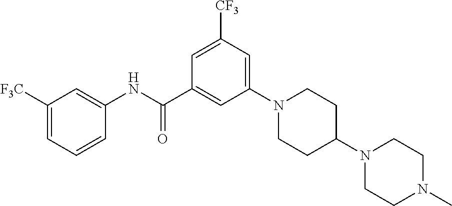 | MS m/z 515.2 (M + 1) |
| 78 | 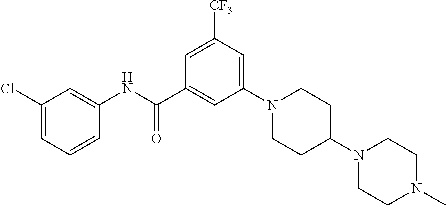 | MS m/z 481.2 (M + 1) ¹H NMR (CD₃OD, 400 MHz) 8.05 (1H, s), 7.85 (1H, d, J = 8.8 Hz), 7.65 (1H, s), 7.56 (1H, s), 7.46 (1H, t, J = 8.0 Hz), 7.34 (1H, d, J = 8.0 Hz), 7.29 (1H, s), 3.90-3.93 (2H, m), 3.27 (4H, br), 3.15 (4H, br), 2.91- 2.99 (1H, m), 2.82-2.89 (2H, m), 2.77 (3H, s), 2.01-2.07 (2H, m), 1.63-1.73 (2H, m). |
| 79 | 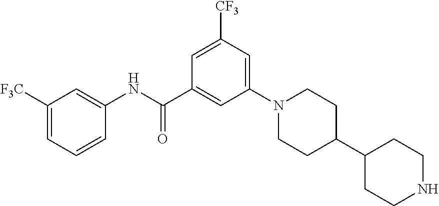 | MS m/z 500.2 (M + 1) |
| 80 | 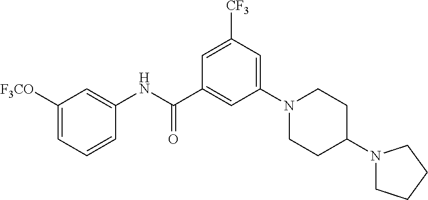 | MS m/z 502.2 (M + 1) |
| 81 | 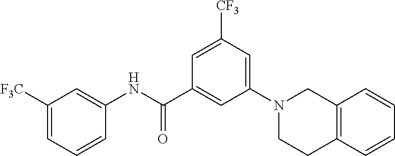 | MS m/z 465.1 (M + 1) ¹H NMR (CD₃OD, 400 MHz) 8.18 (1H, s), 7.98 (1H, d, J = 8.4 Hz), 7.77 (1H, s), 7.60 (1H, s), 7.57 (1H, t, J = 8.0 Hz), 7.46 (1H, d, J = 8.0 Hz), 7.39 (1H, s), 7.26-7.28 (1H, m), 7.20-7.21 (3H, m), 4.57 (2H, s), 3.71 (2H, d, J = 6.0 Hz), 3.04 (2H, d, J = 6.0 Hz). |
| 82 | 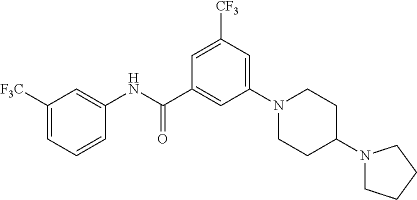 | MS m/z 486.2 (M + 1) ¹H NMR (CDCl₃, 400 MHz) 8.04 (1H, s), 7.97 (1H, s), 7.87 (1H, d, J = 8.0 Hz), 7.59 (1H, s), 7.50 (1H, t, J = 8.0 Hz), 7.42 (1H, d, J = 8.0 Hz), 7.36 (1H, s), 7.25 (1H, s), 3.78- 3.82 (2H, m), 2.88-2.96 (2H, m), 2.59-2.61 (4H, m), 2.17-2.24 (1H, m), 2.01-2.04 (2H, m), 1.79-1.83 (4H, m), 1.71-1.77 (2H, m). |
| 83 | 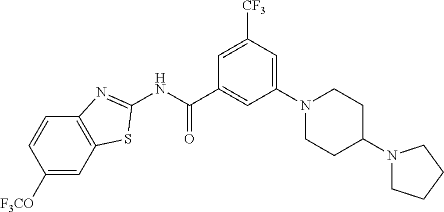 | MS m/z 559.2 (M + 1) |
| 84 | 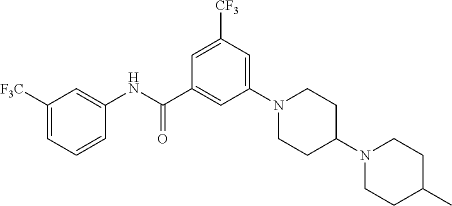 | MS m/z 514.2 (M + 1) ¹H NMR (CD₃OD, 400 MHz) 8.11 (1H, s), 7.90 (1H, d, J = 8.4 Hz), 7.72 (1H, s), 7.65 (1H, s), 7.52 (1H, t, J = 8.0 Hz), 7.41 (1H, t, J = 8.0 Hz), 7.38 (1H, s), 4.04-4.07 (2H, m), 3.53-3.56 (2H, m), 3.32-3.39 (1H, m), 2.99- 3.10 (2H, m), 2.90-2.96 (2H, m), 2.19-2.20 (2H, m), 1.93-1.97 (2H, m), 1.79-1.90 (2H, m), 1.65-1.73 (1H, m), 1.23-1.46 (2H, m), 0.99 (3H, d, J = 6.4 Hz). |
| 85 | 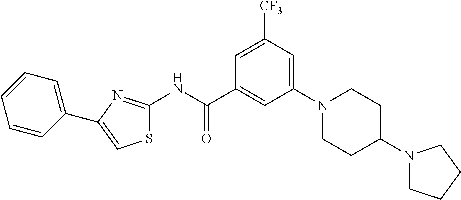 | MS m/z 501.2 (M + 1) |
| 86 | 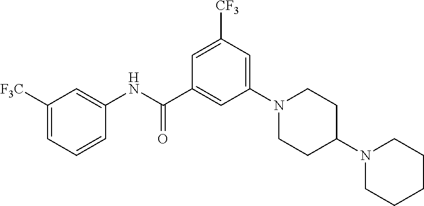 | MS m/z 500.1 (M + 1) ¹H NMR (CDCl₃, 400 MHz) 11.16 (1H, br), 8.71 (1H, s), 8.02 (1H, s), 7.92 (1H, d, J = 8.0 Hz), 7.59 (1H, s), 7.53 (1H, s), 7.49 (1H, t, J = 8.0 Hz), 7.42 (1H, d, J = 8.0 Hz), 7.22 (1H, s), 3.90-3.94 (2H, m), 3.58-3.60 (2H, m), 3.30-3.50 (1H, m), 2.88-2.93 (2H, m), 2.69- 2.77 (2H, m), 2.13-2.18 (2H, m), 1.91-2.01 (8H, m). |
| 87 | 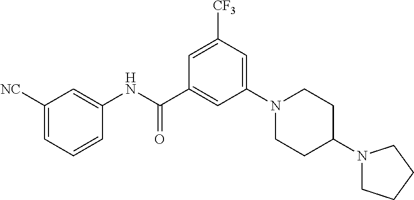 | MS m/z 443.2 (M + 1) |
| 88 | 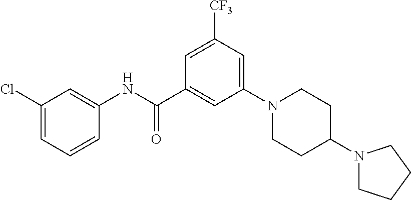 | MS m/z 452.2 (M + 1) ¹H NMR (CD₃OD, 400 MHz) 7.90 (1H, s), 7.76 (1H, s), 7.78 (1H, s), 7.61 (1H, d, J = 8.8 Hz), 7.43 (1H, s), 7.36 (1H, t, J = 8.0 Hz), 7.17 (1H, d, J = 8.0 Hz), 4.05-4.09 (2H, m), 3.68-3.72 (2H, m), 3.34-3.42 (1H, m), 3.17- 3.23 (2H, m), 2.93-3.00 (2H, m), 2.28-2.31 (2H, m), 2.16-2.22 (2H, m), 2.01-2.08 (2H, m), 1.77-1.87 (2H, m). |
| 89 | 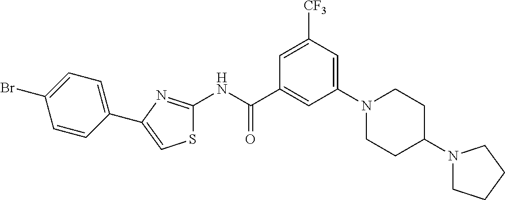 | MS m/z 479.2 (M + 1) |
| 90 | 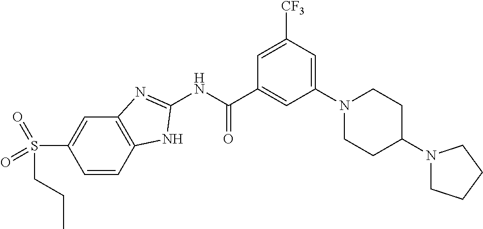 | MS m/z 564.2 (M + 1) |
| 91 | 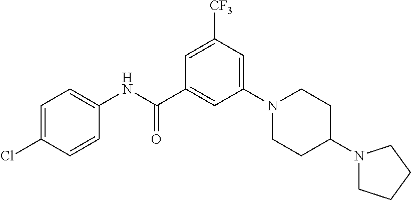 | MS m/z 452.3 (M + 1) |
| 92 | 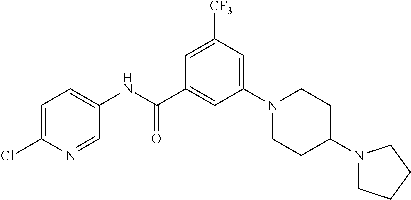 | MS m/z 453.2 (M + 1) |
| 93 | 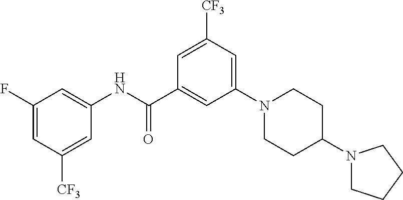 | MS m/z 504.2 (M + 1) |
| 94 | 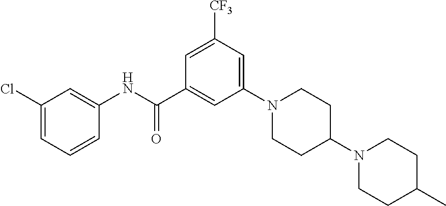 | MS m/z 480.3 (M + 1) |
| 95 | 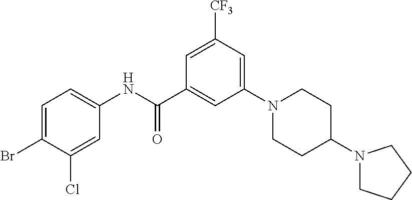 | MS m/z 530.2 (M + 1) |
| 96 | 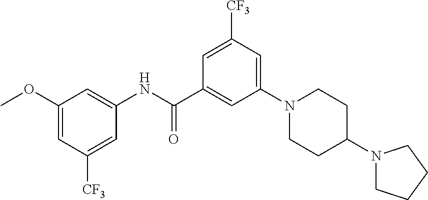 | MS m/z 516.3 (M + 1) |
| 97 | 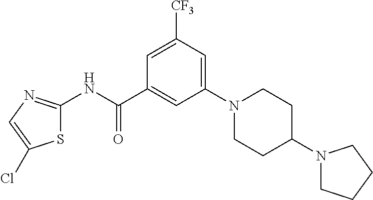 | MS m/z 459.1 (M + 1) |
| 98 | 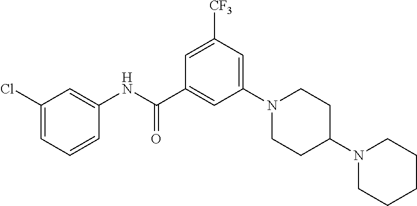 | MS m/z 466.2 (M + 1) ¹H NMR (CDCl₃, 400 MHz) 11.25 (1H, br), 8.35 (1H, s), 7.82 (1H, s), 7.57 (1H, s), 7.54 (1H, d, J = 9.2 Hz), 7.48 (1H, s), 7.30 (1H, t, J = 8.0 Hz), 7.23 (1H, s), 7.15 (1H, d, J = 8.0 Hz), 3.92-3.96 (2H, m), 3.59-3.62 (2H, m), 3.34-3.39 (1H, m), 2.90-2.96 (2H, m), 2.71- 2.78 (2H, m), 2.15-2.18 (2H, m), 1.92-2.07 (8H, m). |
| 99 | 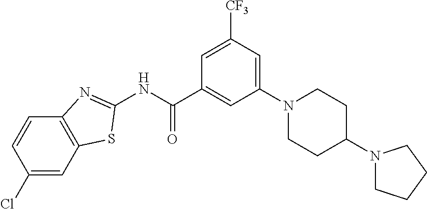 | MS m/z 509.1 (M + 1) |
| 100 | 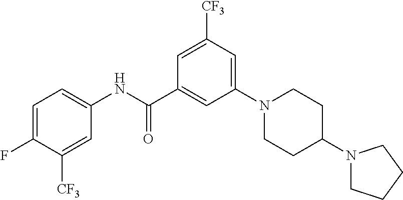 | MS m/z 504.3 (M + 1) |
| 101 | 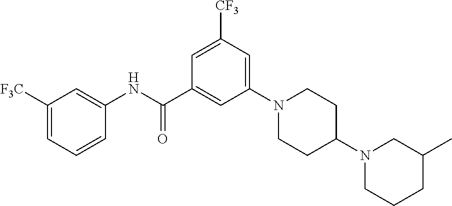 | MS m/z 514.3 (M + 1) ¹H NMR (CD₃OD, 400 MHz) 8.16 (1H, s), 7.95 (1H, d, J = 8.0 Hz), 7.77 (1H, s), 7.69 (1H, s), 7.56 (1H, t, J = 8.0 Hz), 7.45 (1H, d, J = 8.0 Hz), 7.42 (1H, s), 4.08-4.42 (2H, m), 3.56-3.59 (1H, m), 3.38-3.50 (2H, m), 2.94-3.01 (2H, m), 2.64-2.70 (1H, m), 2.22-2.26 (2H, m), 2.01-2.04 (1H, m), 1.87- 1.94 (6H, m), 1.17-1.27 (1H, m), 1.04 (3H, d, J = 6.4 Hz). |
| 102 | 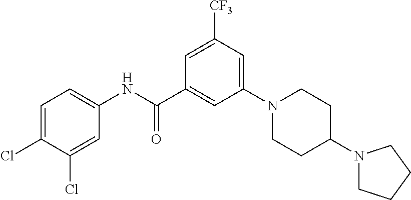 | MS m/z 486.2 (M + 1) |
| 103 | 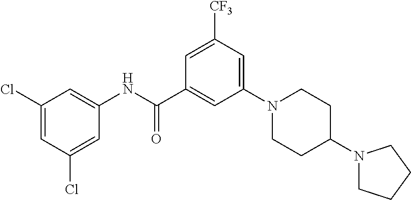 | MS m/z 486.2 (M + 1) |
| 104 | 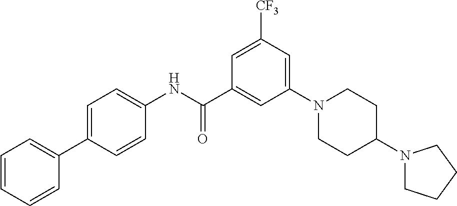 | MS m/z 494.3 (M + 1) ¹H NMR (CDCl₃, 400 MHz) 7.99 (1H, s), 7.72-7.75 (2H, m), 7.59-7.63 (4H, m), 7.72- 7.46 (2H, m), 7.39 (1H, s), 7.32-7.36 (1H, m), 7.24 (1H, s), 3.78-3.82 (2H, m), 2.88-2.94 (2H, m), 2.60-2.63 (4H, m), 2.18-2.23 (1H, m), 2.00-2.04 (2H, m), 1.78-1.85 (4H, m), 1.63-1.72 (2H, m). |
| 105 | 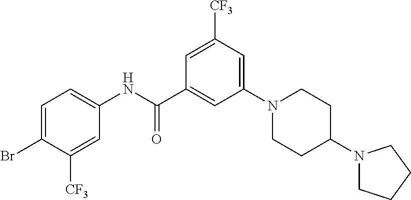 | MS m/z 564.2 (M + 1) |
| 106 | 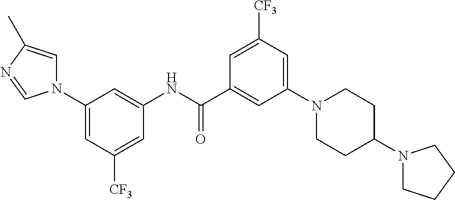 | MS m/z 566.3 (M + 1) ¹H NMR (CDCl₃, 400 MHz) 9.01 (1H, s), 8.21 (1H, s), 7.86 (1H, s), 7.80 (1H, s), 7.63 (1H, s), 7.45 (1H, s), 7.36 (1H, s), 7.25 (1H, s), 7.08 (1H, s), 3.77-3.80 (2H, m), 2.88-2.95 (2H, m), 2.59-2.60 (5H, m), 2.17 (3H, s), 1.95-2.02 (2H, m), 1.78-1.82 (4H, m), 1.60- 1.70 (2H, m). |
| 107 | 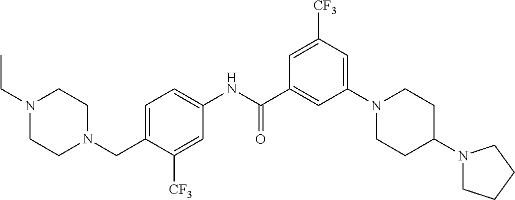 | MS m/z 612.4 (M + 1) |
| 108 | 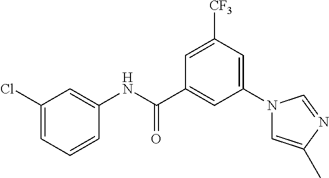 | MS m/z 380.1 (M + 1) |
| 109 | 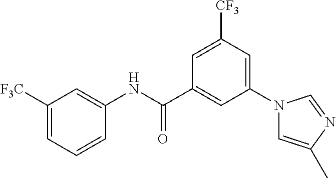 | MS m/z 414.1 (M + 1) |
| 110 | 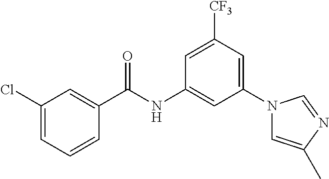 | MS m/z 380.1 (M + 1) |
| 111 | 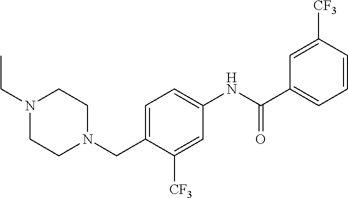 | MS m/z 460.2 (M + 1) |
| 112 | 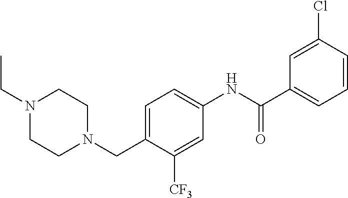 | MS m/z 426.2 (M + 1) |
| 113 | 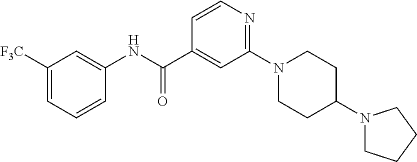 | MS m/z 419.2 (M + 1) |
| 114 | 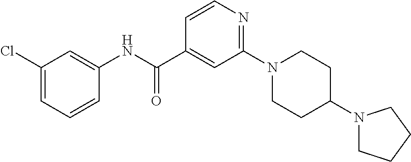 | MS m/z 385.2 (M + 1) |
| 115 | 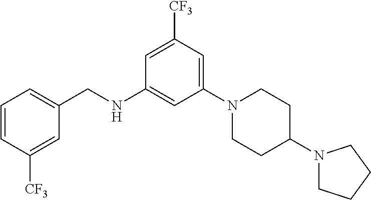 | MS m/z 472.2 (M + 1) ¹H NMR (CD₃OD, 400 MHz) 7.70 (1H, s), 7.67 (1H, s), 7.63 (1H, d, J = 7.2 Hz), 7.60 (1H, s), 7.53-7.57 (2H, m), 7.40 (1H, s), 4.65 (2H, s), 4.01-4.04 (2H, m), 3.68 (2H, br), 3.17 (2H, br), 2.91-2.97 (2H, m), 2.25-2.29 (2H, m), 2.17 (2H, br), 2.02 (2H, br), 1.75-1.85 (2H, m). |
| 116 | 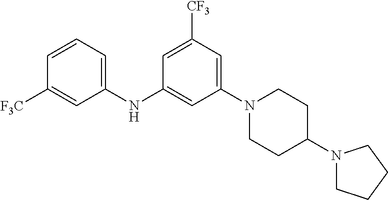 | MS m/z 458.2 (M + 1) |
| 117 | 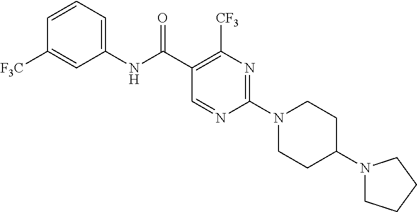 | MS m/z 488.1 (M + 1) ¹H NMR (CD₃OD, 400 MHz) 8.74 (1H, s), 8.06 (1H, s), 7.82 (1H, d, J = 8.4 Hz), 7.56 (1H, t, J = 8.0 Hz), 7.45 (1H, d, J = 8.0 Hz), 5.03-5.06 (2H, m), 3.69 (2H, br), 3.47-3.55 (1H, m), 3.21 (2H, br), 3.06-3.14 (2H, m), 2.29-2.32 (2H, m), 2.19 (2H, br), 2.03 (2H, br), 1.61-1.72 (2H, m). |
| 118 | 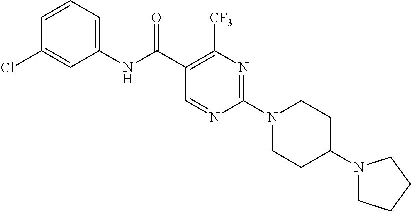 | MS m/z 454.1 (M + 1) |
| 119 | 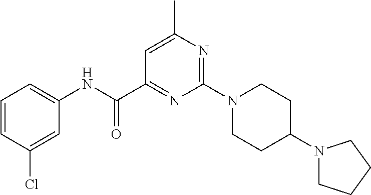 | MS m/z 400.1 (M + 1) |
| 120 | 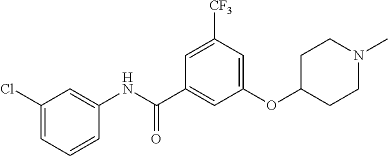 | MS m/z 413.1 (M + 1) |
| 121 | 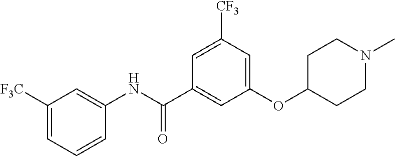 | MS m/z 447.1 (M + 1) ¹H NMR (CDCl₃, 400 MHz) 7.96 (1H, s), 7.86 (1H, d, J = 8.8 Hz), 7.61 (1H, s), 7.52 (1H, t, J = 8.0 Hz), 7.44 (1H, d, J = 7.6 Hz), 7.32 (1H, s), 4.48-4.50 (1H, m), 2.72-2.78 (2H, m), 2.36-2.40 (2H, m), 2.35 (3H, s), 2.01-2.04-2.10 (2H, m), 1.85-1.94 (2H, m). |
| 122 | 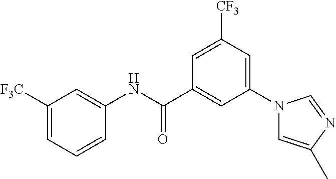 | MS m/z 414.1 (M + 1) |
| 123 | 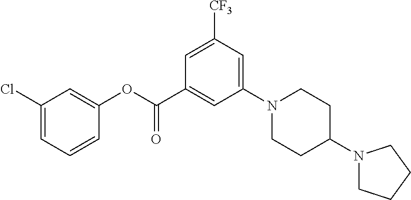 | MS m/z 453.2 (M + 1) |
| 124 | 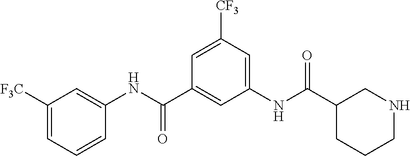 | MS m/z 460.8 (M + 1) |
| 125 | 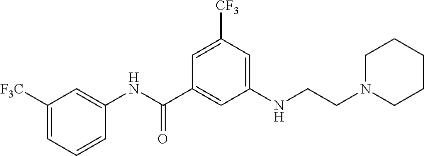 | MS m/z 460.2 (M + 1) |
| 126 | 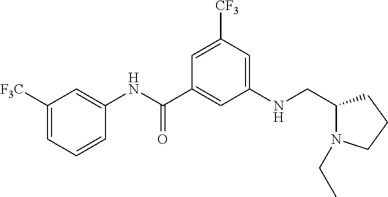 | MS m/z 460.2 (M + 1) |
| 127 | 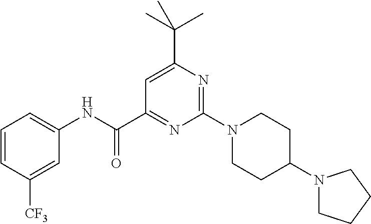 | MS m/z 476.1 (M + 1) |
| 128 | 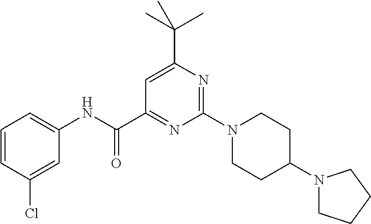 | MS m/z 442.1 (M + 1) ¹H NMR (CD₃OD, 400 MHz) 7.94 (1H, t, J = 2.0 Hz), 7.66 (1H, dq, J = 1.2, 8.4 Hz), 7.40 (1H, s), 7.35 (1H, t, J = 8.0 Hz), 7.17 (1H, dq, J = 1.2, 8.0 Hz), 5.12-5.17 (2H, m), 3.69 (2H, br), 3.44-3.52 (1H, m), 3.20 (2H, br), 2.99- 3.07 (2H, m), 2.26-2.29 (2H, m), 2.17 (2H, br), 2.03 (2H, br), 1.64-1.74 (2H, m), 1.34 (9H, s). |
| Reference Compound 1C | 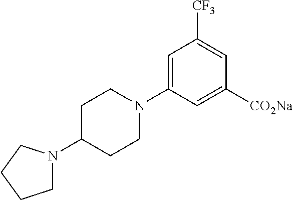 | MS m/z = 343(M + 1, (free acid form). ¹H NMR (CD₃OD, 400 MHz) 7.77 (1H, s), 7.66 (1H, s), 7.27 (1H, s), 3.91-3.94 (2H, m), 3.56-3.63 (1H, m), 3.42 (4H, br), 2.83-2.89 (2H, m), 2.21-2.24 (2H, m), 2.07-2.11 (4H, m), 1.82-1.92 (2H, m). |
| Reference Compound 1D | 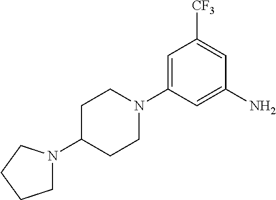 | MS m/z = 314 (M + 1). ¹H NMR (CDCl₃, 400 MHz) 6.54 (1H, s), 6.35 (1H, s), 6.33 (1H, t, J = 2.0 Hz), 3.3.63- 3.68 (2H, m), 2.72-2.79 (2H, m), 2.59-2.62 (4H, br), 2.11-2.18 (1H, m), 1.96-1.99 (2H, m), 1.78-1.82 (4H, m), 1.59-1.69 (2H, m). |
| Reference Compound 2 | 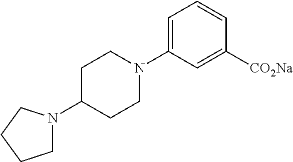 | MS m/z 275.2 (M + 1) (free acid form) |
| Reference Compound 3 | 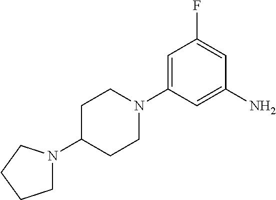 | MS m/z 264.2 (M + 1) ¹H NMR (CD₃OD, 400 MHz) 6.14 (1H, t, J = 2.0 Hz), 6.04 (1H, dt, J = 2.0, 12.4 Hz), 5.93 (1H, dt, J = 2.0, 10.8 Hz), 3.69-3.72 (2H, m), 2.67-2.79 (6H, m), 2.31-2.39 (1H, m), 2.03-2.07 (2H, m), 1.86-1.89 (4H, m), 1.58-1.68 (2H, m). |
| Reference Compound 4 | 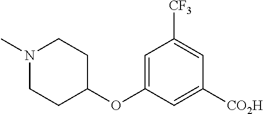 | MS m/z 304.2 (M + 1) |
| Reference Compound 5 | 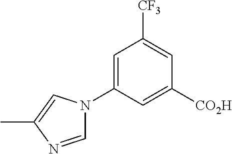 | MS m/z = 312.4 (M + 1) |
| Reference Compound 6 | 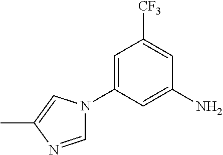 | MS m/z = 283.45 (M + 1) |
| Reference Compound 7 | 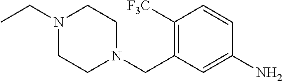 | MS m/z = 421.26 (M + 1). |
| Reference Compound 8 | 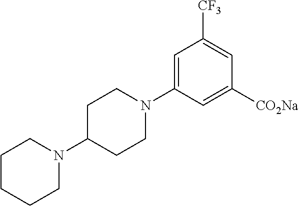 | MS m/z 357.2 (M + 1) (free acid form) ¹H NMR (CD₃OD, 400 MHz) 7.82 (1H, s), 7.69 (1H, s), 7.43 (1H, s), 4.02-4.05 (2H, m), 3.55-3.58 (2H, br), 3.37-3.44 (1H, m), 3.01- 3.07 (2H, m), 2.90-2.96 (2H, m), 2.22 (2H, d, J = 12.4 Hz), 2.00 (2H, d, J = 12.4 Hz), 1.78- 1.90 (6H, m). |
| Reference Compound 9 | 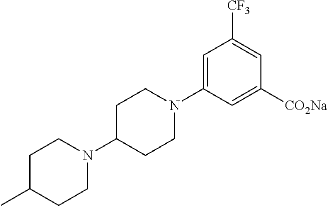 | MS m/z 333.3 (M + 1) (free acid form) |
| Reference Compound 10 | 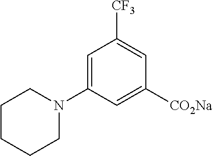 | MS m/z 274.2 (M + 1) (free acid form) |
| Reference Compound 11 | 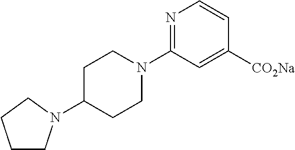 | MS m/z 276.2 (M + 1) (free acid form) ¹H NMR (CD₃OD, 400 MHz) 8.22 (1H, d, J = 5.6 Hz), 7.60 (1H, s), 7.28 (1H, d, J = 5.6 Hz), 4.50-4.53 (2H, m), 3.71 (2H, br), 3.47- 3.53 (1H, m), 3.10-3.21 (4H, m), 2.30-2.33 (2H, m), 2.19 (2H, br), 2.04 (2H, br), 1.74- 1.84 (2H, m). |
| Reference Compound 12 | 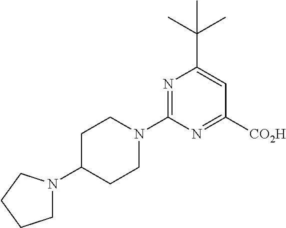 | MS m/z 371.2 (M + 1) |
| Reference Compound 13 | 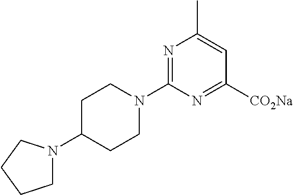 | MS m/z 291.2 (M + 1) (free acid form) ¹H NMR (CD₃OD, 400 MHz) 7.10 (1H, s), 5.04-5.08 (2H, m), 3.66 (2H, br), 3.40-3.48 (1H, m), 3.19 (2H, br), 2.91-2.98 (2H, m), 2.42 (3H, s), 2.20-2.23 (2H, m), 2.10 (4H, br), 1.58-1.68 (2H, m). |
| Reference Compound 14 | 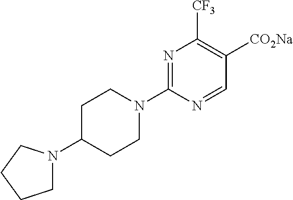 | MS m/z 345.2 (M + 1) (free acid form) |
| Reference Compound 15 | 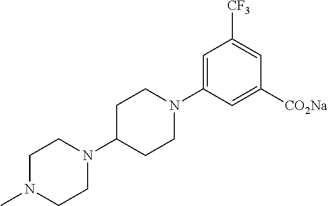 | MS m/z 373.2 (M + 1) (free acid form) ¹H NMR (CD₃OD, 400 MHz) 7.79 (1H, s), 7.67 (1H, s), 7.23 (1H, s), 3.86-3.89 (2H, m), 2.80-2.97 (10H, m), 2.64-2.67 (1H, m), 2.63 (3H, s), 2.00-2.03 (2H, d, J = 12.0 Hz), 1.62- 1.72 (2H, m). |
| Reference Compound 16 | 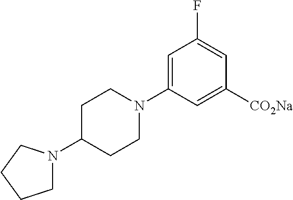 | MS m/z 293.2 (M + 1) (free acid form) ¹H NMR (CD₃OD, 400 MHz) 7.43 (1H, s), 7.13 (1H, d, J = 8.8 Hz), 6.97 (1H, d, J = 12.0 Hz), 3.93-3.96 (2H, m), 3.67 (2H, br), 3.34- 3.38 (1H, m), 3.19 (2H, br), 2.85-2.92 (2H, m), 2.23-2.26 (2H, m), 2.15 (2H, br), 2.04 (2H, br), 1.74-1.84 (2H, m). |
| Reference Compound 17 | 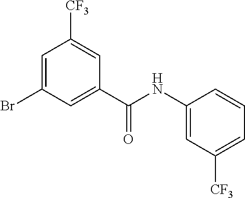 | MS m/z 412.0 (M + 1) |
| Reference Compound 18 | 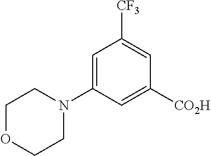 | MS m/z 276.1 (M + 1) |

### Assays

Compounds of the invention can be assayed to measure their capacity to inhibit proliferation of parasitemia in infected red blood cells. The proliferation is quantified by the addition of SYBR Green I (INVITROGEN)® dye which has a high affinity for double stranded DNA.

The following assay illustrates the invention without in any way limiting the scope of the invention.

### Example 129

This parasite proliferation assay measures the increase in parasite DNA content using a DNA intercalating dye, SYBR Green®.

3D7 P. Falciparum strain is grown in complete culturing media until parasitemia reaches 3% to 8% with O+ human erythrocytic cells. 20 μl of screening media is dispensed into 384 well assay plates. A plate containing erythrocytic cells and parasites is included to calculate the baseline and anther plate of erythrocytic cells is included to calculate the background. 50 nl of compounds of the invention (in DMSO), including antimalarial controls (chloroquine and artimesinin), are then transferred into the assay plates. 50 n1 of DMSO is transferred into the baseline and background control plates. Then 30 μl of a suspension of a 3D7 P. falciparum infected erythrocytic cell suspension in screening media is dispensed into the assay plates and the baseline control plate such that the final hematocrit is 2.5% with a final parasitemia of 0.3%. Non-infected erythrocytic cells are dispensed into the background control plate such that the final hematocrit is 2.5%. The plates are placed in a 37° C. incubator for 72 hours in a low oxygen environment containing 93% N₂, 4% CO₂, and 3% O₂ gas mixture. 10 μl of a 10× solution of SYBR Green I® in RPMI media is dispensed into the plates. The plates are sealed and placed in a −80° C. freezer overnight for the lysis of the red blood cells. The plates are thawed, and for optimal staining, left at room temperature overnight. The fluorescence intensity is measured (excitation 497 nm, emission 520 nm) using the ACQUEST™ system (Molecular Devices). The percentage inhibition, EC₅₀, is calculated for each compound.

Compounds of the invention have an EC₅₀ of 10 μM or less, preferably less than 1 μM, 750 nM, 500 nM 400 nM, 300 nM, 200 nM, 100 nM and 50 nM. Compounds of the invention can significantly delay the increase in parasitemia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 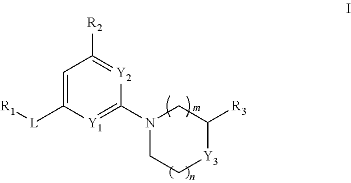
in which
L is selected from —NR₄—, —NR₄S(O)₂—, —S(O)₂NR₄—, —C(O)O—, —OC(O)—, —C(O)—, —NR₄C(O)O—, —OC(O)NR₄—, —NR₄C(O)—, —C(O)NR₄—, —NR₄C(O)NR₄—, —NR₄NR₄C(O)— and —C(O)NR₄NR₄—; wherein R₄ is selected from hydrogen and —SO₂R₅; wherein R₅ is selected from hydrogen and C₁₋₆alkyl;
n and m are independently selected from 0 and 1;
R₁ is selected from C₁₋₆alkyl, C₆₋₁₀aryl-C₀₋₄alkyl, C₃₋₁₂cycloalkyl, 5-10 member heteroaryl and 3-8 member heterocycloalkyl; wherein said heteroaryl and heterocycloalkyl have up to 4 members selected from N, O and S(O)₀₋₂; wherein said aryl, heteroaryl, cycloalkyl or heterocycloalkyl of R₁ is optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy, —NR₆C(O)R₇, —C(O)NR₆R₇, —C(O)OR₇, —S(O)₂NR₆R₇, —S(O)₂R₇, C₆₋₁₀aryl, 3-8 member heterocycloalkyl-C₀₋₄alkyl and 5-10 member heteroaryl; wherein said heteroaryl and heterocycloalkyl have up to 4 members selected from N, O and S(O)₀₋₂; wherein R₆ is selected from hydrogen and C₁₋₆alkyl; and R₇ is selected from hydrogen, C₁₋₆alkyl and 5-10 member heteroaryl; wherein said heteroaryl has up to 4 members selected from N, O and S(O)₀₋₂; wherein said aryl, heterocycloalkyl or heteroaryl substituents of R₁ are optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy and 3-8 member heterocycloalkyl; wherein said heterocycloalkyl has up to 4 members selected from N, O and S(O)₀₋₂; wherein said alkyl substituents of R₁ are optionally substituted with —COOH;
R₂ is selected from hydrogen, halo, C₁₋₆alkyl, halo-substituted-C₁₋₆alkyl, C₁₋₆alkoxy and halo-substituted-C₁₋₆alkoxy;
R₃ is selected from hydrogen, C₁₋₆alkyl, C(O)NR₈R₉ and C(O)OR₉; wherein R₈ and R₉ are independently selected from hydrogen and C₁₋₆alkyl;
Y₁ and Y₂ are independently selected from CH and N;
Y₃ is selected from O, NR₁₀ and CR₁₀R₁₁; wherein R₁₀ and R₁₁ are independently selected from hydrogen, C₁₋₆alkyl, 3-8 member heterocycloalkyl, —NR₁₂R₁₃ and —NR₁₂C(O)OR₁₃; wherein said heterocycloalkyl has up to 4 members selected from N, O and S(O)₀₋₂; wherein said heterocycloalkyl of R₁₀ or R₁₁ is optionally substituted with 1 to 3 radicals independently selected from halo, C₁₋₆alkyl and halo-substituted-C₁₋₆alkyl;
wherein R₁₂ and R₁₃ are independently selected from hydrogen and C₁₋₆alkyl; or R₃ and R₁₀ together with the carbon atoms to which R₃ and R₁₀ are attached from a phenyl ring; and the pharmaceutically acceptable salts thereof.

2. The compound of claim 1 in which:
L is selected from —NR₄—, —S(O)₂NR₄—, —OC(O)—, —OC(O)NR₄—, —NR₄C(O)—, —C(O)NR₄—, —C(O)—, —NR₄C(O)NR₄— and —NR₄NR₄C(O)—; wherein R₄ is selected from hydrogen and —SO₂R₅; wherein R₅ is selected from hydrogen and C₁₋₆alkyl;
n and m are independently selected from 0 and 1;
R₁ is selected from C₁₋₆alkyl, C₆₋₁₀aryl-C₀₋₄alkyl, C₃₋₁₂cycloalkyl, 5-10 member heteroaryl and 3-8 member heterocycloalkyl; wherein said heteroaryl and heterocycloalkyl have up to 4 members selected from N, O and S(O)₀₋₂; wherein said aryl, heteroaryl, cycloalkyl or heterocycloalkyl of R₁ is optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy, —NR₆C(O)R₇, —C(O)NR₆R₇, —C(O)OR₇, —S(O)₂NR₆R₇, —S(O)₂R₇, C₆₋₁₀aryl, 3-8 member heterocycloalkyl-C₀₋₄alkyl and 5-10 member heteroaryl; wherein said heteroaryl and heterocycloalkyl have up to 4 members selected from N, O and S(O)₀₋₂; wherein R₆ is selected from hydrogen and C₁₋₆alkyl; and R₇ is selected from hydrogen, C₁₋₆alkyl and 5-10 member heteroaryl; wherein said heteroaryl has up to 4 members selected from N, O and S(O)₀₋₂; wherein said aryl, heterocycloalkyl or heteroaryl substituents of R₁ are optionally substituted with 1 to 3 radicals independently selected from halo, cyano, C₁₋₆alkyl, halo-substituted-C₁₋₆alkyl, C₁₋₆alkoxy, halo-substituted-C₁₋₆alkoxy and 3-8 member heterocycloalkyl; wherein said heterocycloalkyl has up to 4 members selected from N, O and S(O)₀₋₂; wherein said alkyl substituents of R₁ are optionally substituted with —COOH;
R₂ is selected from hydrogen, halo, C₁₋₆alkyl and halo-substituted-C₁₋₆alkyl;
R₃ is selected from hydrogen, C(O)NR₈R₉ and C(O)OR₉; wherein R₈ and R₉ are independently selected from hydrogen and C₁₋₆alkyl;
Y₁ and Y₂ are independently selected from CH and N;
Y₃ is selected from O, NR₁₀ and CR₁₀R₁₁; wherein R₁₀ and R₁₁ are independently selected from hydrogen, C₁₋₆alkyl, 3-8 member heterocycloalkyl, —NR₁₂R₁₃ and —NR₁₂C(O)OR₁₃; wherein said heterocycloalkyl has up to 4 members selected from N, O and S(O)₀₋₂; wherein said heterocycloalkyl of R₁₀ or R₁₁ is optionally substituted with 1 to 3 radicals independently selected from halo, C₁₋₆alkyl and halo-substituted-C₁₋₆alkyl;
wherein R₁₂ and R₁₃ are independently selected from hydrogen and C₁₋₆alkyl; or R₃ and R₁₀ together with the carbon atoms to which R₃ and R₁₀ are attached from a phenyl ring.

3. The compound of claim 2 in which R₁ is selected from methyl, propyl, phenyl, cyclopropyl, pyridinyl, thiazolyl, pyrimidinyl, indolin-1-yl, piperazinyl, benzyl, 1H-indazol-5-yl, 1H-benzo[d]imidazol-2-yl, imidazolyl, 1H-indol-5-yl, benzo[d]thiazol-2-yl and 4-methyl-2-oxo-1,2-dihydroquinolin-6-yl; wherein said phenyl, benzyl, cyclopropyl, pyridinyl, thiazolyl, N-thiazol-2-ylsulfamoyl, indolin-1-yl, piperazinyl, 1H-indol-5-yl, 1H-indazol-5-yl, 1H-benzo[d]imidazol-2-yl, imidazolyl, benzo[d]thiazol-2-yl or 4-methyl-2-oxo-1,2-dihydroquinolin-6-yl is optionally substituted with 1 to 3 radicals independently selected from halo, cyano, trifluoromethyl, trifluoromethoxy, methyl-carbonyl-amino, amino-carbonyl, methyl, t-butyl, methoxy, propyl-sulfonyl, piperazinyl-methyl, piperidinyl, pyrazolyl, morpholino, imidazolyl, 2-carboxypropan-2-yl, phenyl and ethoxy-carbonyl; wherein said phenyl, piperidinyl, pyrazolyl, morpholino, piperazinyl-methyl or imidazolyl substituents of R₁ are optionally substituted with a radical selected from methyl, trifluoromethyl and pyrrolidinyl.

4. The compound of claim 3 in which R₂ is selected from hydrogen, chloro, fluoro, trifluoromethyl, methyl and t-butyl; and R₃ is selected from amino-carbonyl and ethoxy-carbonyl.

5. The compound of claim 4 in which Y₃ is selected from O, NR₁₀ and CR₁₀R₁₁; wherein R₁₀ is selected from hydrogen and methyl; and R₁₁ is selected from dimethyl-amino, t-butoxy-carbonyl-amino, morpholino, pyrrolidinyl, piperidinyl, piperazinyl, 2-oxopyrrolidin-1-yl and 2-oxopiperidin-1-yl; wherein said morpholino, piperazinyl, pyrrolidinyl, piperidinyl, 2-oxopyrrolidin-1-yl or 2-oxopiperidin-1-yl is optionally substituted with a radical selected from halo and methyl.

6. The compound of claim 5 selected from: N-(methylsulfonyl)-N-(3-(4-(pyrrolidin-1-yl)piperidin-1-yl)-5-(trifluoromethyl)phenyl)methanesulfonamide; N-(3-(4-(pyrrolidin-1-yl)piperidin-1-yl)-5-(trifluoromethyl)phenyl)-3-(trifluoromethyl)benzenesulfonamide; 4-methyl-N-(3-(4-(pyrrolidin-1-yl)piperidin-1-yl)-5-(trifluoromethyl)phenyl)benzenesulfonamide; N-(3-(N-(3-(4-(pyrrolidin-1-yl)piperidin-1-yl)-5-(trifluoromethyl)phenyl)sulfamoyl)phenyl)acetamide; 4-tert-butyl-N-(3-(4-(pyrrolidin-1-yl)piperidin-1-yl)-5-(trifluoromethyl)phenyl)benzenesulfonamide; 4-methoxy-N-(3-(4-(pyrrolidin-1-yl)piperidin-1-yl)-5-(trifluoromethyl)phenyl)benzenesulfonamide; 3-chloro-N-(3-fluoro-5-(4-(pyrrolidin-1-yl)piperidin-1-yl)phenyl)benzamide; tert-butyl 3-(4-(pyrrolidin-1-yl)piperidin-1-yl)-5-(trifluoromethyl)phenylcarbamate; N-(3-(4-(pyrrolidin-1-yl)piperidin-1-yl)-5-(trifluoromethyl)phenyl)cyclopropanecarboxamide; 2-chloro-N-(3-(4-(pyrrolidin-1-yl)piperidin-1-yl)-5-(trifluoromethyl)phenyl)nicotinamide; 2-fluoro-N-(3-(4-(pyrrolidin-1-yl)piperidin-1-yl)-5-(trifluoromethyl)phenyl)benzamide; N-(3-fluoro-5-(4-(pyrrolidin-1-yl)piperidin-1-yl)phenyl)-3-(trifluoromethyl)benzamide; 2,4-dichloro-N-(3-(4-(pyrrolidin-1-yl)piperidin-1-yl)-5-(trifluoromethyl)phenyl)benzamide; 3-cyano-N-(3-(4-(pyrrolidin-1-yl)piperidin-1-yl)-5-(trifluoromethyl)phenyl)benzamide; 4-methoxy-N-(3-(4-(pyrrolidin-1-yl)piperidin-1-yl)-5-(trifluoromethyl)phenyl)benzamide; 3-methyl-N-(3-(4-(pyrrolidin-1-yl)piperidin-1-yl)-5-(trifluoromethyl)phenyl)benzamide; N-(3-(4-(pyrrolidin-1-yl)piperidin-1-yl)-5-(trifluoromethyl)phenyl)-3-(trifluoromethyl)benzamide; 3-fluoro-N-(3-(4-(pyrrolidin-1-yl)piperidin-1-yl)-5-(trifluoromethyl)phenyl)benzamide; 3-(4-(pyrrolidin-1-yl)piperidin-1-yl)-N-(3-(4-(pyrrolidin-1-yl)piperidin-1-yl)-5-(trifluoromethyl)phenyl)-5-(trifluoromethyl)benzamide; 1-(4-chloro-2-methylphenyl)-3-(3-(4-(pyrrolidin-1-yl)piperidin-1-yl)-5-(trifluoromethyl)phenyl)urea; 1-(3-(4-(pyrrolidin-1-yl)piperidin-1-yl)-5-(trifluoromethyl)phenyl)-3-(3-(trifluoromethyl)phenyl)urea; 1-(3-chlorophenyl)-3-(3-(4-(pyrrolidin-1-yl)piperidin-1-yl)-5-(trifluoromethyl)phenyl)urea; 1-(3-(4-methyl-1H-imidazol-1-yl)-5-(trifluoromethyl)phenyl)-3-(3-(4-(pyrrolidin-1-yl)piperidin-1-yl)-5-(trifluoromethyl)phenyl)urea; 1-(3,5-dichlorophenyl)-3-(3-(4-(pyrrolidin-1-yl)piperidin-1-yl)-5-(trifluoromethyl)phenyl)urea; 1,3-bis(3-(4-(pyrrolidin-1-yl)piperidin-1-yl)-5-(trifluoromethyl)phenyl)urea; 2-methyl-2-(4-(3-(4-methyl-1,4′-bipiperidin-1′-yl)-5-(trifluoromethyl)benzamido)phenyl)propanoic acid; 3-(4-(pyrrolidin-1-yl)piperidin-1-yl)-5-(trifluoromethyl)-N-(4-(trifluoromethyl)thiazol-2-yl)benzamide; 3-chloro-N-(3-morpholino-5-(trifluoromethyl)phenyl)benzamide; 3-(4-(pyrrolidin-1-yl)piperidin-1-yl)-N-(4-(N-thiazol-2-ylsulfamoyl)phenyl)-5-(trifluoromethyl)benzamide; 1-(3-(trifluoromethyl)-5-(3-(trifluoromethyl)phenylcarbamoyl)phenyl)piperidine-3-carboxamide; N-propyl-3-(4-(pyrrolidin-1-yl)piperidin-1-yl)-5-(trifluoromethyl)benzamide; N-(3-chlorophenyl)-N-methyl-3-(4-(pyrrolidin-1-yl)piperidin-1-yl)-5-(trifluoromethyl)benzamide; 3-(4-(pyrrolidin-1-yl)piperidin-1-yl)-5-(trifluoromethyl)-N-(6-(trifluoromethyl)pyrimidin-4-yl)benzamide; N-(2-chloropyridin-4-yl)-3-(4-(pyrrolidin-1-yl)piperidin-1-yl)-5-(trifluoromethyl)benzamide; 3-morpholino-5-(trifluoromethyl)-N-(3-(trifluoromethyl)phenyl)benzamide; N-(3-carbamoylphenyl)-3-(4-(pyrrolidin-1-yl)piperidin-1-yl)-5-(trifluoromethyl)benzamide; N-(4-(2-chlorophenyl)thiazol-2-yl)-3-(4-(pyrrolidin-1-yl)piperidin-1-yl)-5-(trifluoromethyl)benzamide; N′-(3-chlorophenyl)-3-(4-(pyrrolidin-1-yl)piperidin-1-yl)-5-(trifluoromethyl)benzohydrazide; 3-(4-(pyrrolidin-1-yl)piperidin-1-yl)-5-(trifluoromethyl)-N′-(3-(trifluoromethyl)phenyl)benzohydrazide; N-(3-chlorophenyl)-3-(piperidin-1-yl)-5-(trifluoromethyl)benzamide; N-(pyrimidin-4-yl)-3-(4-(pyrrolidin-1-yl)piperidin-1-yl)-5-(trifluoromethyl)benzamide; (3-(4-(pyrrolidin-1-yl)piperidin-1-yl)-5-(trifluoromethyl)phenyl)(6-(trifluoromethyl)indolin-1-yl)methanone; N-(2-chlorophenyl)-3-(4-(pyrrolidin-1-yl)piperidin-1-yl)-5-(trifluoromethyl)benzamide; N-(3-chlorophenyl)-3-(4-(pyrrolidin-1-yl)piperidin-1-yl)benzamide; 3-(trifluoromethyl)-N-(3-(trifluoromethyl)phenyl)-5-(4-(3-(trifluoromethyl)phenyl)piperazin-1-yl)benzamide; 3-(piperidin-1-yl)-5-(trifluoromethyl)-N-(3-(trifluoromethyl)phenyl)benzamide; 3-(2-oxo-1,4′-bipiperidin-1′-yl)-5-(trifluoromethyl)-N-(3-(trifluoromethyl)phenyl)benzamide; N-(4-tert-butylthiazol-2-yl)-3-(4-(pyrrolidin-1-yl)piperidin-1-yl)-5-(trifluoromethyl)benzamide; (3-(4-(pyrrolidin-1-yl)piperidin-1-yl)-5-(trifluoromethyl)phenyl)(4-(3-(trifluoromethyl)phenyl)piperazin-1-yl)methanone; tert-butyl 1-(3-(trifluoromethyl)-5-(3-(trifluoromethyl)phenylcarbamoyl)phenyl)piperidin-4-ylcarbamate; N-(4,5-dimethylthiazol-2-yl)-3-(4-(pyrrolidin-1-yl)piperidin-1-yl)-5-(trifluoromethyl)benzamide; N-(3-chloro-4-methoxyphenyl)-3-(4-(pyrrolidin-1-yl)piperidin-1-yl)-5-(trifluoromethyl)benzamide; N-(4-morpholinophenyl)-3-(4-(pyrrolidin-1-yl)piperidin-1-yl)-5-(trifluoromethyl)benzamide; 3-(4-(2-oxopyrrolidin-1-yl)piperidin-1-yl)-5-(trifluoromethyl)-N-(3-(trifluoromethyl)phenyl)benzamide; N-(4-morpholinophenyl)-3-(4-(pyrrolidin-1-yl)piperidin-1-yl)-5-(trifluoromethyl)benzamide; 3-(4-(pyrrolidin-1-yl)piperidin-1-yl)-5-(trifluoromethyl)-N-(3-(trifluoromethyl)benzyl)benzamide; N-(3-(1H-pyrazol-4-yl)phenyl)-3-(4-(pyrrolidin-1-yl)piperidin-1-yl)-5-(trifluoromethyl)benzamide; N-(1H-indazol-5-yl)-3-(4-(pyrrolidin-1-yl)piperidin-1-yl)-5-(trifluoromethyl)benzamide; ethyl 1-(3-(trifluoromethyl)-5-(3-(trifluoromethyl)phenylcarbamoyl)phenyl)piperidine-3-carboxylate; N-phenyl-3-(4-(pyrrolidin-1-yl)piperidin-1-yl)-5-(trifluoromethyl)benzamide; 3-fluoro-5-(4-(pyrrolidin-1-yl)piperidin-1-yl)-N-(3-(trifluoromethyl)phenyl)benzamide; 3-(4-(pyrrolidin-1-yl)piperidin-1-yl)-N-(3-(trifluoromethyl)phenyl)benzamide; ethyl 2-(3-(4-(pyrrolidin-1-yl)piperidin-1-yl)-5-(trifluoromethyl)benzamido)-4-(trifluoromethyl)thiazole-5-carboxylate; N-(1H-benzo[d]imidazol-2-yl)-3-(4-(pyrrolidin-1-yl)piperidin-1-yl)-5-(trifluoromethyl)benzamide; 3-(3-(dimethylamino)pyrrolidin-1-yl)-5-(trifluoromethyl)-N-(3-(trifluoromethyl)phenyl)benzamide; 3-(4-(pyrrolidin-1-yl)piperidin-1-yl)-5-(trifluoromethyl)-N-(1-(3-(trifluoromethyl)phenyl)cyclopropyl)benzamide; N-(4,5-dicyano-1H-imidazol-2-yl)-3-(4-(pyrrolidin-1-yl)piperidin-1-yl)-5-(trifluoromethyl)benzamide; N-(1H-indol-5-yl)-3-(4-(pyrrolidin-1-yl)piperidin-1-yl)-5-(trifluoromethyl)benzamide; 3-(4-morpholinopiperidin-1-yl)-5-(trifluoromethyl)-N-(3-(trifluoromethyl)phenyl)benzamide; N-(3,5-di-tert-butylphenyl)-3-(4-(pyrrolidin-1-yl)piperidin-1-yl)-5-(trifluoromethyl)benzamide; N-(5-phenylthiazol-2-yl)-3-(4-(pyrrolidin-1-yl)piperidin-1-yl)-5-(trifluoromethyl)benzamide; 3-(3-(piperidin-1-yl)pyrrolidin-1-yl)-5-(trifluoromethyl)-N-(3-(trifluoromethyl)phenyl)benzamide; 3-(4-(pyrrolidin-1-yl)piperidin-1-yl)-5-(trifluoromethyl)-N-(4-(trifluoromethyl)phenyl)benzamide; 3-(4-(pyrrolidin-1-yl)piperidin-1-yl)-N-(4-(trifluoromethoxy)phenyl)-5-(trifluoromethyl)benzamide; N-(3,5-bis(trifluoromethyl)phenyl)-3-(4-(pyrrolidin-1-yl)piperidin-1-yl)-5-(trifluoromethyl)benzamide; N-(4-methyl-2-oxo-1,2-dihydroquinolin-6-yl)-3-(4-(pyrrolidin-1-yl)piperidin-1-yl)-5-(trifluoromethyl)benzamide; N-(4-(4-chlorophenyl)thiazol-2-yl)-3-(4-(pyrrolidin-1-yl)piperidin-1-yl)-5-(trifluoromethyl)benzamide; 3-(4-(4-methylpiperazin-1-yl)piperidin-1-yl)-5-(trifluoromethyl)-N-(3-(trifluoromethyl)phenyl)benzamide; N-(3-chlorophenyl)-3-(4-(4-methylpiperazin-1-yl)piperidin-1-yl)-5-(trifluoromethyl)benzamide; 3-(4,4′-bipiperidin-1-yl)-5-(trifluoromethyl)-N-(3-(trifluoromethyl)phenyl)benzamide; 3-(4-(pyrrolidin-1-yl)piperidin-1-yl)-N-(3-(trifluoromethoxy)phenyl)-5-(trifluoromethyl)benzamide; 3-(3,4-dihydroisoquinolin-2(1H)-yl)-5-(trifluoromethyl)-N-(3-(trifluoromethyl)phenyl)benzamide; 3-(4-(pyrrolidin-1-yl)piperidin-1-yl)-N-(6-(trifluoromethoxy)benzo[d]thiazol-2-yl)-5-(trifluoromethyl)benzamide; 3-(4-(pyrrolidin-1-yl)piperidin-1-yl)-5-(trifluoromethyl)-N-(3-(trifluoromethyl)phenyl)benzamide; 3-(4-methyl-1,4′-bipiperidin-1′-yl)-5-(trifluoromethyl)-N-(3-(trifluoromethyl)phenyl)benzamide; N-(4-phenylthiazol-2-yl)-3-(4-(pyrrolidin-1-yl)piperidin-1-yl)-5-(trifluoromethyl)benzamide; 3-(1,4′-bipiperidin-1′-yl)-5-(trifluoromethyl)-N-(3-(trifluoromethyl)phenyl)benzamide; N-(3-cyanophenyl)-3-(4-(pyrrolidin-1-yl)piperidin-1-yl)-5-(trifluoromethyl)benzamide; N-(3-chlorophenyl)-3-(4-(pyrrolidin-1-yl)piperidin-1-yl)-5-(trifluoromethyl)benzamide; N-(4-(4-bromophenyl)thiazol-2-yl)-3-(4-(pyrrolidin-1-yl)piperidin-1-yl)-5-(trifluoromethyl)benzamide; N-(5-(propylsulfonyl)-1H-benzo[d]imidazol-2-yl)-3-(4-(pyrrolidin-1-yl)piperidin-1-yl)-5-(trifluoromethyl)benzamide; N-(4-chlorophenyl)-3-(4-(pyrrolidin-1-yl)piperidin-1-yl)-5-(trifluoromethyl)benzamide; N-(6-chloropyridin-3-yl)-3-(4-(pyrrolidin-1-yl)piperidin-1-yl)-5-(trifluoromethyl)benzamide; N-(3-fluoro-5-(trifluoromethyl)phenyl)-3-(4-(pyrrolidin-1-yl)piperidin-1-yl)-5-(trifluoromethyl)benzamide; N-(4-bromo-3-chlorophenyl)-3-(4-(pyrrolidin-1-yl)piperidin-1-yl)-5-(trifluoromethyl)benzamide; N-(3-chlorophenyl)-3-(4-methyl-1,4′-bipiperidin-1′-yl)-5-(trifluoromethyl)benzamide; N-(5-chlorothiazol-2-yl)-3-(4-(pyrrolidin-1-yl)piperidin-1-yl)-5-(trifluoromethyl)benzamide; N-(3-methoxy-5-(trifluoromethyl)phenyl)-3-(4-(pyrrolidin-1-yl)piperidin-1-yl)-5-(trifluoromethyl)benzamide; 3-(1,4′-bipiperidin-1′-yl)-N-(3-chlorophenyl)-5-(trifluoromethyl)benzamide; N-(6-chlorobenzo[d]thiazol-2-yl)-3-(4-(pyrrolidin-1-yl)piperidin-1-yl)-5-(trifluoromethyl)benzamide; 3-(3-methyl-1,4′-bipiperidin-1′-yl)-5-(trifluoromethyl)-N-(3-(trifluoromethyl)phenyl)benzamide; N-(4-fluoro-3-(trifluoromethyl)phenyl)-3-(4-(pyrrolidin-1-yl)piperidin-1-yl)-5-(trifluoromethyl)benzamide; N-(3,4-dichlorophenyl)-3-(4-(pyrrolidin-1-yl)piperidin-1-yl)-5-(trifluoromethyl)benzamide; N-(3-(4-methyl-1H-imidazol-1-yl)-5-(trifluoromethyl)phenyl)-3-(4-(pyrrolidin-1-yl)piperidin-1-yl)-5-(trifluoromethyl)benzamide; N-(3,5-dichlorophenyl)-3-(4-(pyrrolidin-1-yl)piperidin-1-yl)-5-(trifluoromethyl)benzamide; N-(biphenyl-4-yl)-3-(4-(pyrrolidin-1-yl)piperidin-1-yl)-5-(trifluoromethyl)benzamide; N-(4-((4-ethylpiperazin-1-yl)methyl)-3-(trifluoromethyl)phenyl)-3-(4-(pyrrolidin-1-yl)piperidin-1-yl)-5-(trifluoromethyl)benzamide; N-(4-bromo-3-(trifluoromethyl)phenyl)-3-(4-(pyrrolidin-1-yl)piperidin-1-yl)-5-(trifluoromethyl)benzamide; 2-(4-(pyrrolidin-1-yl)piperidin-1-yl)-N-(3-(trifluoromethyl)phenyl)isonicotinamide; N-(3-chlorophenyl)-2-(4-(pyrrolidin-1-yl)piperidin-1-yl)isonicotinamide; 3-(4-(pyrrolidin-1-yl)piperidin-1-yl)-5-(trifluoromethyl)-N-(3-(trifluoromethyl)phenyl)aniline; 3-(4-(pyrrolidin-1-yl)piperidin-1-yl)-5-(trifluoromethyl)-N-(3-(trifluoromethyl)benzyl)aniline; 2-(4-(pyrrolidin-1-yl)piperidin-1-yl)-4-(trifluoromethyl)-N-(3-(trifluoromethyl)phenyl)pyrimidine-5-carboxamide; N-(3-chlorophenyl)-2-(4-(pyrrolidin-1-yl)piperidin-1-yl)-4-(trifluoromethyl)pyrimidine-5-carboxamide; N-(3-chlorophenyl)-6-methyl-2-(4-(pyrrolidin-1-yl)piperidin-1-yl)pyrimidine-4-carboxamide; 6-tert-butyl-2-(4-(pyrrolidin-1-yl)piperidin-1-yl)-N-(3-(trifluoromethyl)phenyl)pyrimidine-4-carboxamide; and 6-tert-butyl-N-(3-chlorophenyl)-2-(4-(pyrrolidin-1-yl)piperidin-1-yl)pyrimidine-4-carboxamide.

7. A compound selected from: N-(3-chlorophenyl)-3-(4-methyl-1H-imidazol-1-yl)-5-(trifluoromethyl)benzamide; 3-(4-methyl-1H-imidazol-1-yl)-5-(trifluoromethyl)-N-(3-(trifluoromethyl)phenyl)benzamide; 3-chloro-N-(3-(4-methyl-1H-imidazol-1-yl)-5-(trifluoromethyl)phenyl)benzamide; N-(4-((4-ethylpiperazin-1-yl)methyl)-3-(trifluoromethyl)phenyl)-3-(trifluoromethyl)benzamide; N-(3-chlorophenyl)-3-(1-methylpiperidin-4-yloxy)-5-(trifluoromethyl)benzamide; 3-chloro-N-(4-((4-ethylpiperazin-1-yl)methyl)-3-(trifluoromethyl)phenyl)benzamide; 3-(1-methylpiperidin-4-yloxy)-5-(trifluoromethyl)-N-(3-(trifluoromethyl)phenyl)benzamide; 3-(4-methyl-1H-imidazol-1-yl)-5-(trifluoromethyl)-N-(3-(trifluoromethyl)phenyl)benzamide; N-(3-(trifluoromethyl)-5-(3-(trifluoromethyl)phenylcarbamoyl)phenyl)piperidine-3-carboxamide; 3-(2-(piperidin-1-yl)ethylamino)-5-(trifluoromethyl)-N-(3-(trifluoromethyl)phenyl)benzamide; (S)-3-((1-ethylpyrrolidin-2-yl)methylamino)-5-(trifluoromethyl)-N-(3-(trifluoromethyl)phenyl)benzamide; and the pharmaceutically acceptable salts thereof.

8. A method for treating a Plasmodium related disease in a subject to prevent, inhibit or ameliorate the pathology and/or symptamology of the Plasmodium related disease, comprising administering to a subject a therapeutically effective amount of a compound of claim 6 or claim 7 and optionally in combination with a second agent.

9. The method of claim 8 wherein the Plasmodium related disease is malaria.

10. The method of claim 9, wherein the contacting occurs in vitro or in vivo.

11. The method of claim 10, wherein the second agent is selected from a kinase inhibitor, an anti-malarial drug and an anti-inflammatory agent.

12. The method of claim 11 wherein the anti-malarial drug is selected from proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, and pyronaridine.

13. The method of claim 12, wherein the compound of claim 1 or claim 7 is administered prior to, simultaneously with, or after the second agent.

14. The method of claim 13, wherein said subject is a human.
